(19) United States
(12) Patent Application Publication
(10) Pub. No.: US 2025/0169706 A1
(43) Pub. Date: May 29, 2025

(54) ENDOSCOPE SYSTEM, OPERATION METHOD FOR ENDOSCOPE SYSTEM, AND NON-TRANSITORY COMPUTER READABLE MEDIUM

(71) Applicant: FUJIFILM Corporation, Tokyo (JP)

(72) Inventors: Yu YOSHIMORI, Kanagawa (JP); Kenichi OTANI, Kanagawa (JP)

(73) Assignee: FUJIFILM Corporation, Tokyo (JP)

(21) Appl. No.: 18/959,637

(22) Filed: Nov. 26, 2024

(30) Foreign Application Priority Data
Nov. 28, 2023 (JP) 2023-201000

(51) Int. Cl. A61B 5/026; A61B 1/00; A61B 5/00

(52) U.S. Cl. CPC A61B 5/026; A61B 1/000094; A61B 5/0084; A61B 5/7425; A61B 5/743; A61B 5/7435

(57) ABSTRACT

In a blood volume mode, at least one of a blood volume image or a blood-volume-and-oxygen-saturation-at-each-time-point graph is displayed on an extended display. An image acquisition unit acquires a reference point image and acquires a blood volume mode image at a time interval set by a user. A calculation unit calculates average values of a blood volume and an oxygen saturation based on pixels included in a region of interest. A time-series image generation unit generates the blood volume image showing a change amount of the blood volume. A temporal change graph generation unit generates the blood-volume-and-oxygen-saturation-at-each-time-point graph for the time points at which the images are acquired.

### CROSS-REFERENCE TO RELATED APPLICATIONS

This application claims priority under 35 U.S.C § 119(a) to Japanese Patent Application No. 2023-201000 filed on 28 Nov. 2023. The above application is hereby expressly incorporated by reference, in its entirety, into the present application.

### BACKGROUND OF THE INVENTION

### 1. Field of the Invention

The present invention relates to an endoscope system having an oxygen saturation imaging function, an operation method for the endoscope system, and a non-transitory computer readable medium.

### 2. Description of the Related Art

In a surgical operation, for example, in a case of resecting cancer, not only a main lesion part but also a blood vessel or a lymph node that may have an invasive or metastatic potential is removed. Therefore, in a stage where the cancer is resected, a part of a vascular network that nourishes the organ itself is also lost, and hemodynamics of the reconstructed organ is likely to be unstable. In particular, in a case where a blood flow at an anastomotic site is poor, there is a possibility that a serious complication of suture insufficiency will occur after the surgery, and it is necessary to evaluate blood perfusion of the reconstructed organ during the surgery.

In particular, in a surgical region, congestion is widely recognized as an effective index for preventing suture insufficiency, and it is generally important to ascertain and avoid this state. For checking of blood perfusion, an endoscope system having a function of calculating an amount of hemoglobin as an amount of a colorant and displaying a concentration distribution with emphasis is known (JP3328627B). In addition, an endoscope system having a function of measuring a blood volume and an oxygen saturation in any region of interest is known (JP2810718B).

### SUMMARY OF THE INVENTION

It is considered that a blood volume in a congestion state transitions in state over time from immediately after treatment of a blood vessel. Therefore, it is required not only to be able to check the congestion state at a certain time point (immediately after a vascular treatment or the like) but also to be able to check a change in a blood retention state over time. However, in a case of performing the evaluation of the blood perfusion as described above, it has been difficult to check temporal transition of the congestion state during the surgery.

An object of the present invention is to provide an endoscope system, an operation method for the endoscope system, and a non-transitory computer readable medium, which can ascertain a degree of progression of congestion by visualizing a blood volume via special light imaging and continuously recording a temporal change image from a certain reference point.

According to an exemplary embodiment of the invention, there is provided an endoscope system comprising a processor, in which the processor is configured to: execute image acquisition processing of acquiring an endoscopic image obtained by imaging an observation target at a time interval set in advance by a user; execute calculation processing of calculating a blood volume and an oxygen saturation for each image acquired in the image acquisition processing; generate an image showing a change amount of the blood volume for each image acquired in the calculation processing with reference to a blood volume at a time point set by the user; generate a graph showing temporal changes of the blood volume and the oxygen saturation for each image acquired in the calculation processing; and display at least one of the image showing the change amount of the blood volume or the graph showing the temporal changes of the blood volume and the oxygen saturation on a display.

It is preferable that the processor is configured to perform reference point imaging of setting the observation target that is a reference as a region of interest in the image acquisition processing.

It is preferable that the processor is configured to calculate average values of the blood volume and the oxygen saturation based on pixels included in the region of interest, and perform control of setting the calculated average values as reference values of the blood volume and the oxygen saturation.

It is preferable that the processor is configured to perform subtraction processing of subtracting the reference value of the blood volume from the blood volume calculated in the calculation processing.

It is preferable that the processor is configured to assign a color map with different shades according to the change amount with reference to the blood volume calculated in the subtraction processing, and generate an image in which the color map is superimposed on the image.

It is preferable that the processor is configured to generate an image in which the change amount is superimposed on the image by representing a minus-side change and a plus-side change with different colors with reference to the blood volume calculated in the subtraction processing.

It is preferable that the processor is configured to perform control of executing the calculation processing based on pixels in the same region as the observation target set as the region of interest for each image acquired in the image acquisition processing to generate the graph in which the calculated blood volume and oxygen saturation are plotted, in displaying the graph.

It is preferable that the processor is configured to perform control of converting the image obtained by the image acquisition processing into a thumbnail image and displaying the image in parallel in time series on the display.

It is preferable that the processor is configured to perform control of displaying a scroll bar for scrolling through the images displayed in parallel on the display.

It is preferable that the processor is configured to perform control of enabling the user to change display or non-display in the graph.

It is preferable that the processor is configured to perform pattern matching based on a blood vessel shape of the observation target in the reference point imaging, and perform control of executing registration in a case where an endoscope or the observation target moves.

It is preferable that the processor is configured to perform control of notifying the user of operation guidance for resetting the region of interest in a case of setting the observation target, which is affected by a disturbance, as the region of interest in the reference point imaging.

According to another aspect of the exemplary embodiment of the invention, there is provided an operation method for an endoscope system including a processor, the method comprising: via the processor, a step of executing image acquisition processing of acquiring an endoscopic image obtained by imaging an observation target at a time interval set in advance by a user; a step of executing calculation processing of calculating a blood volume and an oxygen saturation for each image acquired in the image acquisition processing; a step of generating an image showing a change amount of the blood volume for each image acquired in the calculation processing with reference to a blood volume at a time point set by the user; a step of generating a graph showing temporal changes of the blood volume and the oxygen saturation for each image acquired in the calculation processing; and a step of displaying at least one of the image showing the change amount of the blood volume or the graph showing the temporal changes of the blood volume and the oxygen saturation on a display.

According to still another aspect of the exemplary embodiment of the invention, there is provided a non-transitory computer readable medium for storing a computer-executable program for causing a computer to function as an endoscope system, the computer-executable program causing a computer to implement: a function of executing image acquisition processing of acquiring an endoscopic image obtained by imaging an observation target at a time interval set in advance by a user; a function of executing calculation processing of calculating a blood volume and an oxygen saturation for each image acquired in the image acquisition processing; a function of generating an image showing a change amount of the blood volume for each image acquired in the calculation processing with reference to a blood volume at a time point set by the user; a function of generating a graph showing temporal changes of the blood volume and the oxygen saturation for each image acquired in the calculation processing; and a function of displaying at least one of the image showing the change amount of the blood volume or the graph showing the temporal changes of the blood volume and the oxygen saturation on a display.

According to the exemplary embodiments of the invention, it is possible to ascertain a degree of progression of congestion by visualizing a blood volume via special light imaging and continuously recording a temporal change image from a certain reference point.

### BRIEF DESCRIPTION OF THE DRAWINGS

FIG. 1 is a schematic diagram of an endoscope system for a digestive tract.

FIGS. 2A and 2B are explanatory diagrams showing display aspects on a display and an extended display in a normal mode.

FIGS. 3A and 3B are explanatory diagrams showing display aspects on a display and an extended display in a blood volume mode.

FIG. 4A is an image diagram of an extended display that displays a digestive tract internal blood volume image, and FIG. 4B is an image diagram of an extended display that displays a serosal side blood volume image.

FIG. 5 is a block diagram showing functions of the endoscope system.

FIG. 6 is a table showing illumination and image signals to be acquired in the normal mode.

FIG. 7 is a table showing illumination and image signals to be acquired in the blood volume mode.

FIG. 8 is a block diagram showing functions of an extended processor device.

FIG. 9 is a block diagram showing functions of a blood volume image processing unit.

(A) of FIG. 10 is an explanatory diagram showing an operation of aligning a region-of-interest target with a congestion site in reference point imaging, and (B) of FIG. 10 is an explanatory diagram illustrating setting of a region of interest in the reference point imaging.

FIG. 11A is a graph showing a reflection spectrum of hemoglobin that is different depending on a blood concentration, and FIG. 11B is a graph illustrating a signal ratio in which reflected light in each wavelength region is standardized.

FIG. 12 is a graph showing a change in blood concentration and showing contour lines representing an oxygen saturation.

FIG. 13A is an explanatory diagram showing control of performing calculation processing based on pixels included in an entire image in subtraction processing, and FIG. 13B is an explanatory diagram showing control of performing the calculation processing based on pixels included in a region of interest of a reference point image in the subtraction processing.

FIG. 14 is an explanatory diagram illustrating a series of controls for generating an image showing a change amount of a blood volume.

(A) of FIG. 15 is an explanatory diagram showing an image before superimposing the change amount of the blood volume, and (B) of FIG. 15 is an explanatory diagram showing an image in which the change amount of the blood volume is shown by superimposing a color map with different shades.

FIG. 16A is an explanatory diagram showing an image in which the change amount of the blood volume is superimposed by representing a minus-side change and a plus-side change with different colors, and FIG. 16B is an explanatory diagram showing an image in which the change amount of the blood volume is shown by superimposing a color map with different colors and different shades.

(A) of FIG. 17 is an explanatory diagram showing control of performing calculation processing based on pixels included in a region of interest of an image acquired at each time point, and (B) of FIG. 17 is a graph in which the blood volume and the oxygen saturation obtained by the calculation processing are plotted for each time point.

FIG. 18A is an explanatory diagram of a case where an image in which the change amount of the blood volume is superimposed and a graph showing the blood volume and the oxygen saturation at each time point are displayed on the extended display, and FIG. 18B is an explanatory diagram of a case where an endoscopic image and a graph showing the blood volume and the oxygen saturation at each time point are displayed on the extended display.

FIG. 19 is an explanatory diagram in a case where only an image in which the change amount of the blood volume is superimposed is displayed on the extended display.

(A) of FIG. 20 is an explanatory diagram showing an example in which a normal site is set as a region of interest, (B) of FIG. 20 is an explanatory diagram showing a case where a doubtful site is a congestion site, and (C) of FIG. 20 is an explanatory diagram showing a case where a doubtful site has the same blood volume as the normal site.

FIG. 21 is a flowchart showing a flow of a series of processing in a case where Example 1 is performed in the blood volume mode.

(A) of FIG. 22 is an explanatory diagram showing an example in which the congestion site is set as the region of interest, (B) of FIG. 22 is an explanatory diagram of a case where an image in which the change amount of the blood volume is superimposed and a graph showing the blood volume and the oxygen saturation are displayed on the extended display, and (C) of FIG. 22 is an explanatory diagram of a case where an image in which the change amount of the blood volume changed over time is superimposed and a graph showing a temporal change of the blood volume and the oxygen saturation are displayed on the extended display.

FIG. 23 is a flowchart showing a flow of a series of processing of an example in which the congestion site in the blood volume mode is set as the region of interest.

(A) of FIG. 24 is an explanatory diagram showing an example in which blood vessel processing is performed by setting the congestion site as the region of interest, (B) of FIG. 24 is an explanatory diagram of a case where an image in which the change amount of the blood volume is superimposed and a graph showing the blood volume and the oxygen saturation are displayed on the extended display by using a blood vessel processing site as a target, and (C) of FIG. 24 is an explanatory diagram of a case where an image in which the change amount of the blood volume changed over time is superimposed and a graph showing a temporal change of the blood volume and the oxygen saturation are displayed on the extended display by using a blood vessel processed site as a target.

FIG. 25 is a block diagram showing functions of a parallel display processing unit of a blood volume image display unit in Example 3.

(A) of FIG. 26 is an explanatory diagram of a case where a past image is converted into a thumbnail image and displayed on the extended display, (B) of FIG. 26 is an explanatory diagram in which the past image converted into the thumbnail image is enlarged, and (C) of FIG. 26 is an explanatory diagram of a case where the past image converted into the thumbnail image is selected and displayed on the extended display.

FIG. 27A is an explanatory diagram showing a case where the past image converted into the thumbnail image and a scroll bar are displayed on the extended display, FIG. 27B is an explanatory diagram showing a case where the scroll bar transitions to the right, and FIG. 27C is an explanatory diagram showing a case where the scroll bar transitions to a right end.

FIG. 28 is a block diagram showing functions of a pattern matching unit of Modification Example 1 of the blood volume image display unit in Example 3.

(A) of FIG. 29 is an explanatory diagram illustrating a case where the congestion site is set as the region of interest and the reference point imaging is performed, (B) of FIG. 29 is an explanatory diagram showing a case where a blood vessel shape captured in the reference point imaging is subjected to pattern matching, and (C) of FIG. 29 is an explanatory diagram showing the pattern matching in a case where a position or a distance between an endoscope and an observation target is changed.

FIG. 30 is a block diagram showing functions of a notification unit of Modification Example 2 of the blood volume image display unit in Example 3.

(A) of FIG. 31 is an explanatory diagram of a case where fat is set as the region of interest in the reference point imaging, and (B) of FIG. 31 is an explanatory diagram illustrating a display that notifies of operation guidance in a case where the observation target affected by a disturbance is set as the region of interest.

### DESCRIPTION OF THE PREFERRED EMBODIMENTS

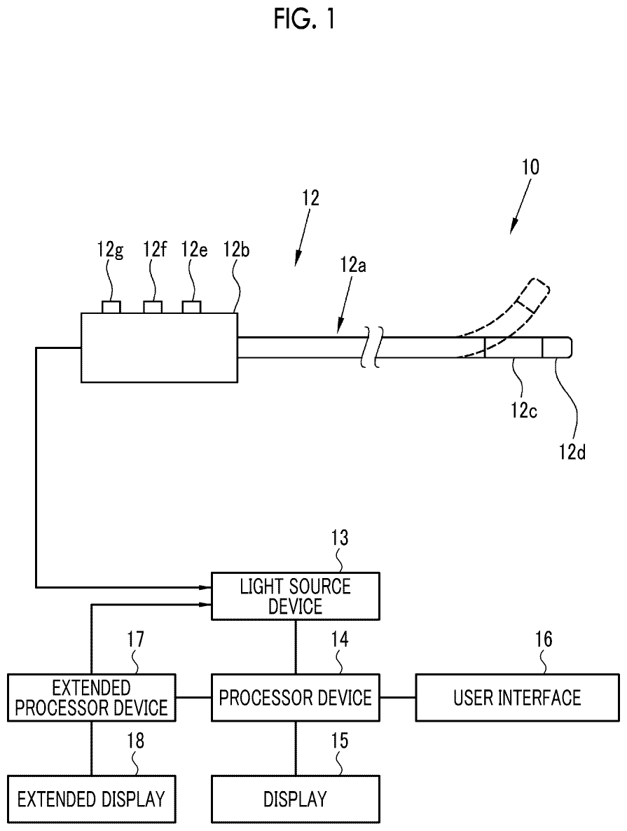

As shown in FIG. 1, an endoscope system 10 comprises an endoscope 12, a light source device 13, a processor device 14, a display 15, a user interface 16, an extended processor device 17, and an extended display 18. The endoscope 12 is optically or electrically connected to the light source device 13 and electrically connected to the processor device 14. The extended processor device 17 is electrically connected to the light source device 13 and the processor device 14. It should be noted that a “display” in the claims includes the extended display 18 in addition to the display 15.

The endoscope 12 has an insertion part 12a, an operating part 12b, a bendable part 12c, and a tip part 12d. The insertion part 12a is inserted into a body of a subject. The operating part 12b is provided at a base end portion of the insertion part 12a. The bendable part 12c and the tip part 12d are provided on a tip side of the insertion part 12a. The tip part 12d is directed in a desired direction by a bending operation of the bendable part 12c. A forceps channel (not shown) for inserting a treatment tool or the like is provided from the insertion part 12a to the tip part 12d.

An optical system for forming a subject image and an optical system for irradiating the subject with illumination light are provided inside the endoscope 12. The operating part 12b is provided with a mode selector switch 12e, a reference point image acquisition instruction switch 12f, and a zoom operation part 12g. The mode selector switch 12e is used for an observation mode switching operation. The reference point image acquisition instruction switch 12f is used to issue an instruction to acquire an image of a subject used as a reference in a blood volume mode described below. The zoom operation part 12g is used for an operation of enlarging or reducing an observation target.

The light source device 13 generates illumination light. The processor device 14 performs system control of the endoscope system 10, and further generates an image obtained by the endoscope (hereinafter, referred to as an endoscopic image) by performing image processing or the like on an image signal transmitted from the endoscope 12. The display 15 displays a medical image transmitted from the processor device 14. The user interface 16 includes a keyboard, a mouse, a microphone, a tablet terminal, a foot switch, a touch pen, and the like, and receives an input operation such as function setting.

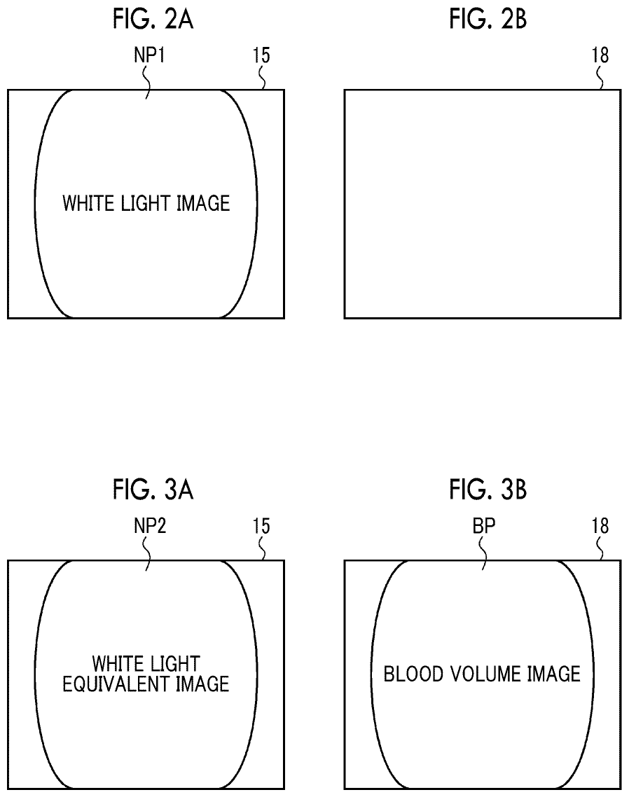

The endoscope system 10 has two modes of a normal mode and a blood volume mode, and these two modes are switched in a case where a user operates the mode selector switch 12e. As shown in FIGS. 2A and 2B, in the normal mode, while a natural color white light image obtained by imaging an observation target using white light as the illumination light is displayed on the display 15, nothing is displayed on the extended display 18.

As shown in FIGS. 3A and 3B, in the blood volume mode, a blood volume and an oxygen saturation of the observation target are calculated at a time interval set in advance by the user, and temporal changes of the calculated blood volume and oxygen saturation are displayed on the extended display 18 as a blood volume image. In addition, in the blood volume mode, a white light equivalent image NP2 having less short-wavelength components than a white light image NP1 is displayed on the display 15.

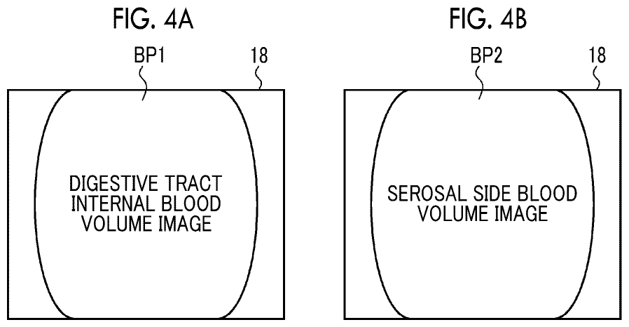

The endoscope system 10 is a soft endoscope type for a digestive tract such as a stomach and a large intestine, and in the blood volume mode, as shown in FIG. 4A, the endoscope system 10 displays a digestive tract internal oxygen saturation image, which is an image of an oxygen saturation state inside the digestive tract, on the extended display 18. In addition, in a case of a rigid endoscope type for an abdominal cavity such as a serosa, as shown in FIG. 4B, a serosal side oxygen saturation image, which is an image of an oxygen saturation state on a serosal side, is displayed on the extended display 18. It is preferable that, as the serosal side oxygen saturation image, an image in which chroma saturation is adjusted with respect to the white light equivalent image is used. It is preferable that the adjustment of the chroma saturation is performed in the blood volume mode regardless of distinction between a mucosa, the serosa, the soft endoscope, and the rigid endoscope.

In the blood volume mode, the blood volume and the oxygen saturation can be accurately calculated in the following cases.
In a case where a predetermined target site (for example, an esophagus, a stomach, or a large intestine) is observed
In a case other than an outer body environment with ambient lighting
In a case where no residue, residual liquid, mucus, blood, or fat remains on the mucosa or the serosa
In a case where a colorant is not sprayed onto the mucosa
In a case where the endoscope 12 is separated from the observation site by more than 7 mm
In a case where the observation site is observed at an appropriate distance without the endoscope being greatly separated from the observation site
Region in which sufficient illumination light is incident
In a case where there is little specular reflection light from the observation site
Region inside ⅔ of the oxygen saturation image
In a case where movement of the endoscope is small or movement of a patient, such as pulsation or breathing, is small
In a case where a blood vessel in a deep part of a digestive tract mucosa is not observed

The processor device 14 is electrically connected to the display 15 and the user interface 16. The processor device 14 receives an image signal from the endoscope 12, and performs various types of processing based on the image signal. The display 15 outputs and displays an image, information, or the like of the observation target that is processed by the processor device 14. The user interface 16 includes a keyboard, a mouse, a touch pad, a microphone, a foot pedal, and the like, and has a function of receiving an input operation such as function setting.

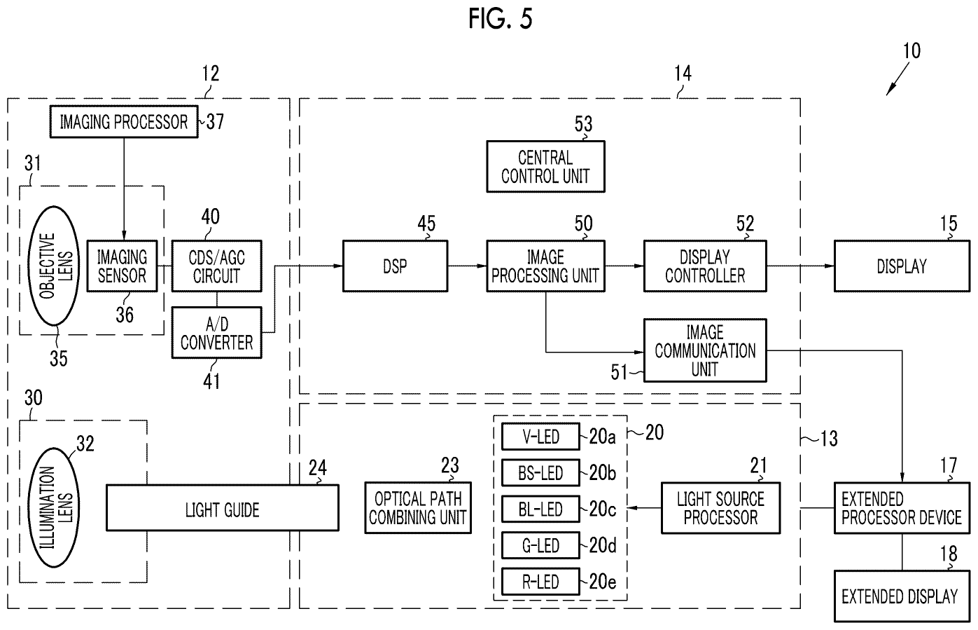

As shown in FIG. 5, the light source device 13 comprises a light source unit 20 and a light source processor 21 that controls the light source unit 20. The light source unit 20 includes, for example, a plurality of semiconductor light sources, turns on or off each of these semiconductor light sources, and emits illumination light, which illuminates the observation target, by controlling an amount of light emitted from each semiconductor light source, in a case where each semiconductor light source is turned on. In the present embodiment, the light source unit 20 includes five color LEDs including a violet light-emitting diode (V-LED) 20a, a blue short-wavelength light-emitting diode (BS-LED) 20b, a blue long-wavelength light-emitting diode (BL-LED) 20c, a green light-emitting diode (G-LED) 20d, and a red light-emitting diode (R-LED) 20e.

The V-LED 20a emits violet light V of 410 nm±10 nm. The BS-LED 20b emits second blue light BS of 450 nm±10 nm. The BL-LED 20c emits first blue light BL of 470 nm #10 nm. The G-LED 20d emits green light G in a green band. It is preferable that a central wavelength of the green light G is 550 nm. The R-LED 20e emits red light R in a red band. It is preferable that a central wavelength of the red light R is 620 nm. A central wavelength and a peak wavelength of each of the LEDs 20a to 20e may be the same or may be different from each other.

The light source processor 21 independently controls turning-on or turning-off of each of the LEDs 20a to 20e, the amount of light emitted in a case of turning-on, and the like by independently inputting a control signal to each of the LEDs 20a to 20e. The control of the turning-on or turning-off in the light source processor 21 differs depending on each mode.

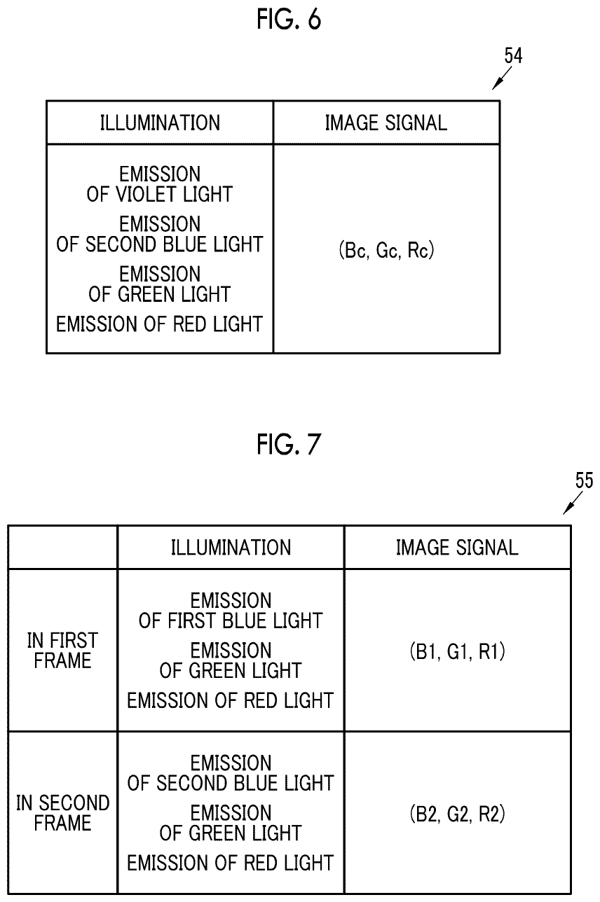

The processor device 14 performs control of the light emission in the normal mode via the light source processor 21. As shown in FIG. 6, in a normal mode 54, as the V-LED 20a, the BS-LED 20b, the G-LED 20d, and the R-LED 20e are simultaneously turned on, white light is emitted that includes the violet light V having a central wavelength of 410 nm, the second blue light BS having a central wavelength of 450 nm, the green light G having a wide band in the green band, and the red light R having a central wavelength of 620 nm. The white light image NP1 obtained based on the white light is displayed on the display 15 (see FIGS. 2A and 2B).

The processor device 14 performs control of the light emission in the blood volume mode via the light source processor 21. As shown in FIG. 7, in a blood volume mode 55, the light emission for two frames having different light emission patterns is repeatedly performed. In a first frame, as the BL-LED 20c, the G-LED 20d, and the R-LED 20e are simultaneously turned on, first illumination light is emitted that has a wide band including the first blue light BL having a central wavelength of 470 nm, the green light G having a wide band in the green band, and the red light R having a central wavelength of 620 nm. In a second frame, as the BS-LED 20b, the G-LED 20d, and the R-LED 20e are simultaneously turned on, second illumination light is emitted that includes the second blue light BS having a central wavelength of 450 nm, the green light G having a wide band in the green band, and the red light R having a central wavelength of 620 nm. The white light equivalent image NP2 obtained based on the light emission of the second illumination light of the second frame is displayed on the display 15 (see FIGS. 3A and 3B). In addition, a blood volume image OP obtained based on the emission of the first illumination light of the first frame and the second illumination light of the second frame is displayed on the extended display 18.

Light emitted from each of the LEDs 20a to 20e is incident onto a light guide 24 via an optical path combining unit 23 composed of a mirror, a lens, and the like. The light guide 24 is built in the endoscope 12 and a universal cord (a cord connecting the endoscope 12, the light source device 13, and the processor device 14). The light guide 24 propagates the light from the optical path combining unit 23 to the tip part 12d of the endoscope 12.

The tip part 12d of the endoscope 12 is provided with an illumination optical system 30 and an imaging optical system 31. The illumination optical system 30 has an illumination lens 32, and the observation target is irradiated with illumination light, which is propagated by the light guide 24, via the illumination lens 32. The imaging optical system 31 has an objective lens 35 and an imaging sensor 36. Light from the observation target, which is irradiated with the illumination light, is incident onto the imaging sensor 36 via the objective lens 35. Accordingly, an image of the observation target is formed on the imaging sensor 36.

The imaging sensor 36 is a color imaging sensor that images the observation target which is being illuminated with the illumination light. Each pixel of the imaging sensor 36 is provided with any one of a blue pixel (B pixel) having a blue (B) color filter, a green pixel (G pixel) having a green (G) color filter, or a red pixel (R pixel) having a red (R) color filter. Spectral transmittances of the B color filter, the G color filter, and the R color filter will be described below. For example, the imaging sensor 36 is preferably a color imaging sensor of a Bayer array in which a ratio of the number of pixels of the B pixels, the G pixels, and the R pixels is 1:2:1.

A charge-coupled device (CCD) imaging sensor or a complementary metal-oxide semiconductor (CMOS) imaging sensor can be used as the imaging sensor 36. In addition, a complementary color imaging sensor comprising complementary color filters corresponding to cyan (C), magenta (M), yellow (Y), and green (G) may be used instead of the primary color imaging sensor 36. In a case where the complementary color imaging sensor is used, image signals corresponding to four colors of C, M, Y, and G are output. Therefore, by converting the image signals corresponding to the four colors of C, M, Y, and G into image signals corresponding to three colors of R, G, and B through complementary color-primary color conversion, the image signals corresponding to respective colors of R, G, and B which are the same as those of the imaging sensor 36 can be obtained.

The imaging sensor 36 is drive-controlled by an imaging processor 37. A correlated double sampling/automatic gain control (CDS/AGC) circuit 40 performs correlated double sampling (CDS) or automatic gain control (AGC) on an analog image signal obtained from the imaging sensor 36. The image signal that has passed through the CDS/AGC circuit 40 is converted into a digital image signal by an analog/digital (A/D) converter 41. The digital image signal, which has been subjected to A/D conversion, is input to the processor device 14.

In the normal mode 54, the imaging processor 37 controls the imaging sensor 36 such that the observation target illuminated with the violet light V, the second blue light BS, the green light G, and the red light R is imaged for each frame. Accordingly, a Bc image signal is output from the B pixel of the imaging sensor 36, a Gc image signal is output from the G pixel thereof, and an Rc image signal is output from the R pixel thereof (see FIG. 6).

In the blood volume mode 55, in a case where the observation target is illuminated with the first illumination light including the first blue light BL, the green light G, and the red light R in the first frame, the imaging processor 37 outputs a B1 image signal from the B pixel of the imaging sensor 36, outputs a G1 image signal from the G pixel thereof, and outputs an R1 image signal from the R pixel thereof, so that a B1 image, a G1 image, and an R1 image are obtained as a first illumination light image. In a case where the observation target is illuminated with the second illumination light including the second blue light BS, the green light G, and the red light R in the second frame, a B2 image signal is output from the B pixel of the imaging sensor 36, a G2 image signal is output from the G pixel thereof, and an R2 image signal is output from the R pixel thereof, so that a B2 image, a G2 image, and an R2 image are obtained as a second illumination light image (see FIG. 7).

The processor device 14 comprises a digital signal processor (DSP) 45, an image processing unit 50, a display controller 52, and a central control unit 53. In the processor device 14, a program related to various types of processing is incorporated in a program memory (not shown). As the central control unit 53 configured by a processor executes the program in the program memory to implement functions of the DSP 45, the image processing unit 50, the display controller 52, and the central control unit 53 are realized.

The DSP 45 performs various types of signal processing, such as defect correction processing, offset processing, gain correction processing, linear matrix processing, gamma conversion processing, demosaicing processing, white balance processing, YC conversion processing, and noise reduction processing, with respect to an image signal received from the endoscope 12. In the defect correction processing, a signal of a defective pixel of the imaging sensor 36 is corrected. In the offset processing, a dark current component is removed from the image signal on which the defect correction processing is performed, and an accurate zero level is set. In the gain correction processing, a signal level of each image signal is adjusted by multiplying the image signal of each color after the offset processing by a specific gain. The image signal of each color after the gain correction processing is subjected to the linear matrix processing for enhancing color reproducibility.

After that, brightness and chroma saturation of each image signal are adjusted by the gamma conversion processing. The demosaicing processing (also referred to as equalization processing or demosaicing) is performed on the image signals having been subjected to the linear matrix processing, so that signals corresponding to missing colors in the respective pixels are generated by interpolation. All the pixels are made to have signals corresponding to the respective colors of R, G, and B by the demosaicing processing. The DSP 45 performs the YC conversion processing on each image signal after the demosaicing processing, and outputs a brightness signal Y and color difference signals Cb and Cr to the DSP 45. The DSP 45 performs noise reduction processing on the image signal that has been subjected to the demosaicing processing or the like by, for example, a moving average method, a median filter method, or the like.

The image processing unit 50 performs various types of image processing on the image signal from the DSP 45. The image processing includes color conversion processing such as 3×3 matrix processing, gradation transformation processing, and three-dimensional look up table (LUT) processing, and structure enhancement processing such as color enhancement processing and spatial frequency enhancement. The image processing unit 50 performs image processing according to the mode. In a case of the normal mode 54, the image processing unit 50 generates the white light image by performing image processing for the normal mode. In a case of the blood volume mode, the image processing unit 50 generates the white light equivalent image by performing image processing for the oxygen saturation. In addition, in a case of the blood volume mode, the image processing unit 50 transmits the image signal from the DSP 45 to the extended processor device 17 via an image communication unit 51.

The display controller 52 performs display control for displaying image information such as the white light image NP1, the blood volume image BP, or other information from the image processing unit 50, on the display 15. In accordance with the display control, the white light image NP1 or the white light equivalent image NP2 is displayed on the display 15.

The extended processor device 17 receives an image signal from the processor device 14 and performs various types of image processing. The extended processor device 17 calculates the blood volume and the oxygen saturation in the blood volume mode 55, and generates the blood volume image BP in which the calculated blood volume and oxygen saturation are visualized. The generated blood volume image BP is an image showing a change amount of the calculated blood volume and/or a graph showing temporal changes of the calculated blood volume and oxygen saturation. The generated blood volume image BP is displayed on the extended display 18. In addition, in a case of starting the blood volume mode 55, the extended processor device 17 sets the observation target used as a reference in the region of interest via a user operation, and performs the reference point imaging for obtaining reference values for the calculation of the blood volume and the oxygen saturation. Details of the blood volume mode 55 performed by the extended processor device 17 will be described below.

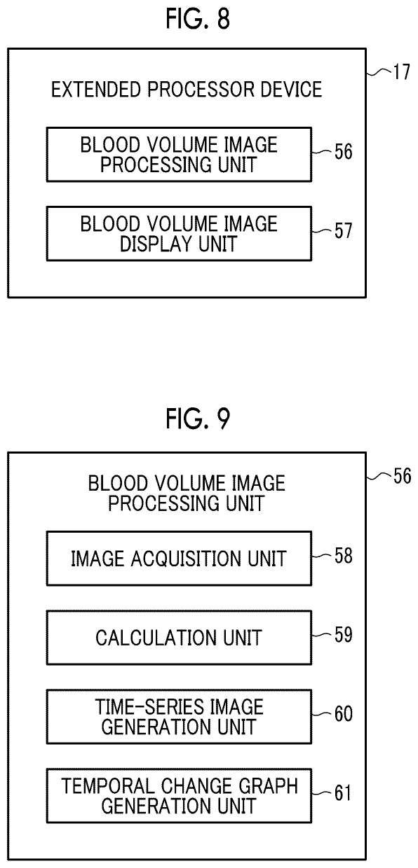

As shown in FIG. 8, the extended processor device 17 comprises a blood volume image processing unit 56 and a blood volume image display unit 57. The blood volume image processing unit 56 performs image processing for generating the blood volume image BP. The blood volume image display unit 57 performs control such as displaying the generated blood volume image BP on the extended display 18.

The switching from the normal mode 54 to the blood volume mode 55 may be performed according to an operation of the mode selector switch 12e by the user, or may be automatically switched according to a condition set by the user. The blood volume image processing unit 56 switches a light emission pattern of the light source processor 21 and a transmission destination of the image signal in response to a mode switching based on the operation or setting of the user.

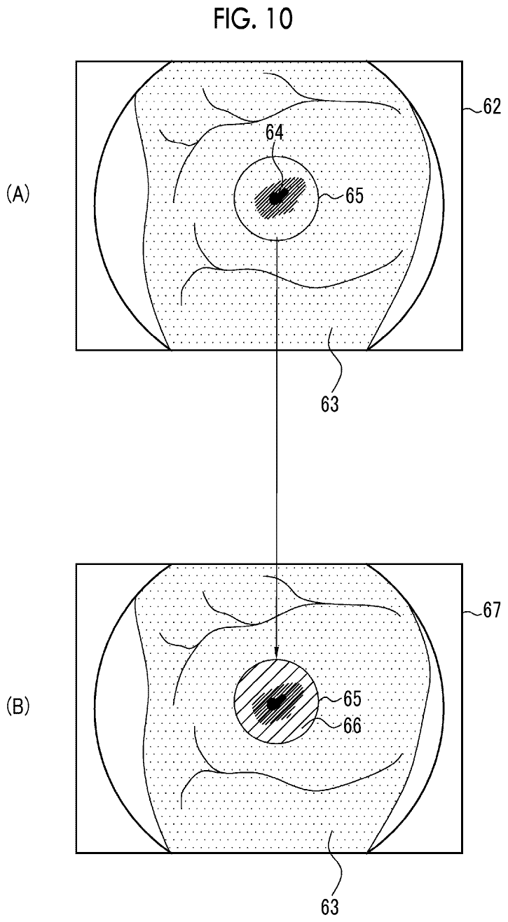

The blood volume mode will be described below. In a case of the blood volume mode, as shown in FIG. 9, the blood volume image processing unit 56 comprises an image acquisition unit 58, a calculation unit 59, a time-series image generation unit 60, and a temporal change graph generation unit 61. The image acquisition unit 58 acquires the first illumination light image via the first illumination light sent from the processor device 14 and the second illumination light image via the second illumination light sent from the processor device 14 in the blood volume mode 55.

The image acquisition unit 58 executes image acquisition processing of acquiring an image obtained by imaging the observation target via the endoscope 12 at a time interval set in advance by the user. The time interval for automatically acquiring the captured image at a certain time interval is set by the user. The time interval may be set or changed at any timing.

The image acquisition unit 58 performs the reference point imaging in which the observation target used as a reference is set as the region of interest. For example, as shown in (A) of FIG. 10, an observation target 63, a congestion site 64, and a region-of-interest target 65 are captured in a blood volume mode image 62 which is any of the first illumination light image or the second illumination light image acquired by the image acquisition unit 58. The blood volume mode image 62 is an example of the endoscopic image acquired by the image acquisition unit 58. In addition, it is preferable that the region-of-interest target 65 is indicated by a solid line having a light color. In addition, a shape of the region-of-interest target 65 is not limited to a circular shape, and may be changed and shown in a rectangular shape or the like, or a size thereof may be changed.

As shown in (B) of FIG. 10, the image acquisition unit 58 performs control of acquiring a reference point image 67 for setting a region including the congestion site 64 as a region of interest 66 in the observation target 63 shown in the blood volume mode image 62. For example, the user fixes the endoscope at a position where at least a part of the congestion site 64, more preferably, an entirety of the congestion site 64, is contained in the region-of-interest target 65, and presses the reference point image acquisition instruction switch 12f to execute the reference point imaging, thereby acquiring the reference point image 67.

The calculation unit 59 executes calculation processing of calculating the blood volume and the oxygen saturation for each image acquired by the image acquisition unit 58. Specifically, for each pixel included in the region of interest 66 (see (B) of FIG. 10) shown in the reference point image 67 acquired by the image acquisition unit 58, the calculation processing of calculating the blood volume and the oxygen saturation and using average values in the region of interest 66 as the reference values of the blood volume and the oxygen saturation is executed. The pixel used for the calculation may be all the pixels included in the region of interest 66, or may be some of the pixels included in the region of interest 66, such as every one pixel or every two pixels. In addition, the reference value is used in a case of calculating the change amount of the blood volume of the observation target.

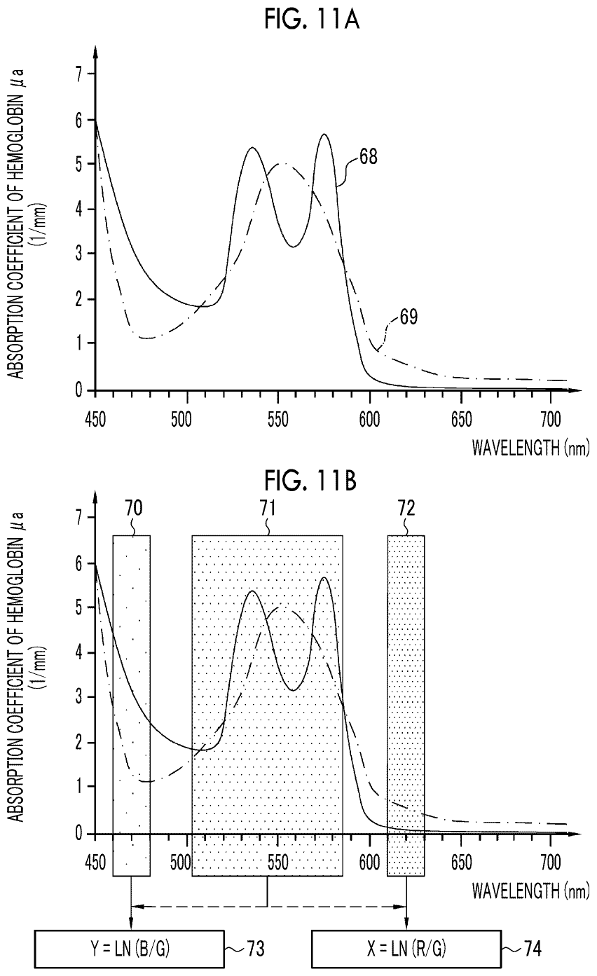

The calculation processing in the blood volume is performed on the pixel included in the image by using a standardized signal ratio in a long-wave region. Specifically, with respect to absorption coefficients of oxidized hemoglobin 68 and reduced hemoglobin 69 at each wavelength as shown in FIG. 11A, reflected light 72 in the vicinity of 620 nm is standardized by reflected light 71 in the vicinity of 550 nm, and the blood volume is calculated using a signal ratio 74 in the long-wave region as shown in FIG. 11B. In addition, since the signal ratio 74 in the long-wave region is affected to a certain extent by a change in the oxygen saturation, the calculation for nullifying the influence is executed. Specifically, the oxygen saturation is calculated using a signal ratio 73 in a short-wave region in which reflected light 70 in the vicinity of 470 nm is standardized by the reflected light 71 in the vicinity of 550 nm. In FIG. 11B, LN represents a natural logarithm.

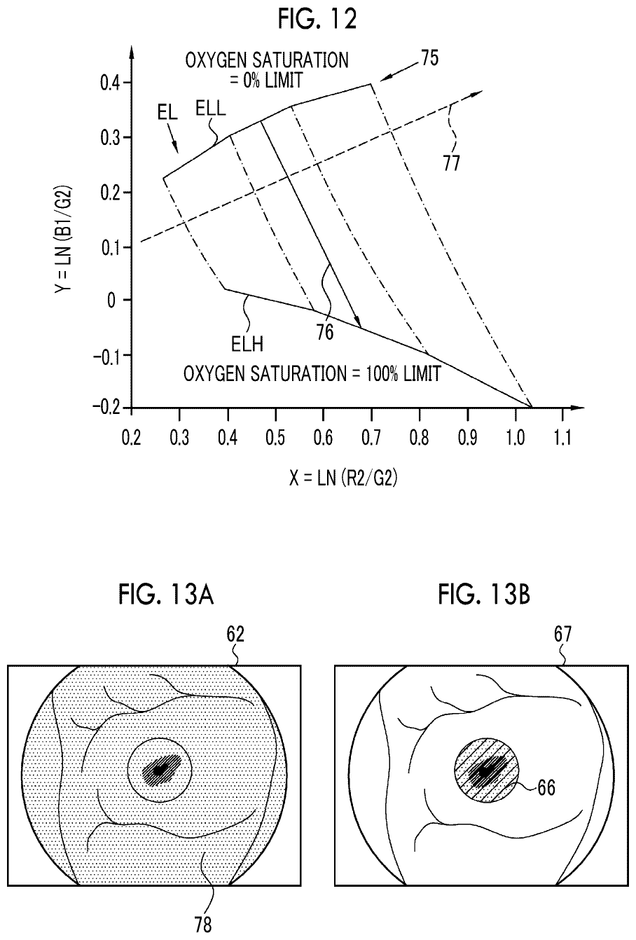

The calculation unit 59 calculates the blood volume by using the signal ratio 74 in the long-wave region and the signal ratio 73 in the short-wave region in the first illumination light image and the second illumination light image (see FIG. 7) acquired by the image acquisition unit 58, and a look-up table (hereinafter, referred to as an LUT). For example, the blood volume is calculated based on an LUT 75 shown in FIG. 12, and the LUT 75 is composed of a change direction 76 of the oxygen saturation and a change direction 77 of the blood volume perpendicular to the change direction 76 of the oxygen saturation. The change direction 76 of the oxygen saturation is represented by a contour line EL, a contour line ELL represents that the oxygen saturation is 0%, and a contour line ELH represents that the oxygen saturation is 100%. The change direction 77 of the blood volume is calculated to be perpendicular to the change direction 76 of the oxygen saturation by giving a certain amount of inclination to a signal ratio LN (R2/G2) in the long-wave region, so that an influence of the oxygen saturation on a signal ratio LN (B1/G2) in the short-wave region is nullified, and only the blood volume is affected. In FIG. 12, LN represents a natural logarithm. In addition, the change amount of the blood volume is calculated by the following Expression (1), where a represents an inclination at which the change direction 77 of the blood volume is perpendicular to the change direction 76 of the oxygen saturation.

$$Change amount of blood volume ( HBI ) = X + α Y \qquad ( 1 )$$

The calculation unit 59 calculates the oxygen saturation of the observation target by using the first illumination light image and the second illumination light image acquired by the image acquisition unit 58, and the LUT (see FIG. 7). Specifically, the calculation unit 59 calculates the ratio LN (B1/G2) between the B1 image based on the B1 image signal and the G2 image based on the G2 image signal and the ratio LN (R2/G2) between the R2 image and the G2 image for each pixel. The ratio LN (B1/G2) and the ratio LN (R2/G2) are reference values calculated by the calculation unit 59 in the reference point image, and are calculation results of the calculation using pixel values in the region of interest in the reference point image. The ratio LN (B1/G2) mainly affects the oxygen saturation, and the ratio LN (R2/G2) mainly affects the blood volume. Therefore, by checking a balance between the ratio LN (B1/G2) and the ratio LN (R2/G2), the oxygen saturation of the observation target can be obtained excluding the influence on the blood volume.

It is rare that the ratio LN (B1/G2) and the ratio LN (R2/G2) are extremely large values or extremely small values. That is, a combination of the ratio LN (B1/G2) and the ratio LN (R2/G2) rarely becomes a combination that exceeds an upper limit contour line representing the oxygen saturation of “100%” or a combination that falls below a lower limit contour line representing the oxygen saturation of “0%”. The calculation unit 59 sets the oxygen saturation to 100% in a case where the oxygen saturation exceeds 100%, and sets the oxygen saturation to 0% in a case where the oxygen saturation is less than 0%.

The calculation unit 59 performs subtraction processing of subtracting the reference value of the blood volume from the blood volume calculated based on the pixels included in the image. For example, as shown in FIG. 13A, in the blood volume mode image 62, the calculation unit 59 calculates the blood volume based on pixels 78 included in the blood volume mode image 62. Further, the calculation unit 59 performs subtraction processing of subtracting the reference value of the blood volume calculated based on the pixels included in the region of interest 66 in the reference point image 67 as shown in FIG. 13B from the blood volume calculated based on the pixels 78 included in the blood volume mode image 62. Therefore, the calculation unit 59 calculates the change amount of the blood volume between the reference value of the blood volume of the reference point image 67 and the blood volume of the observation target captured in the blood volume mode image 62 via the subtraction processing.

The time-series image generation unit 60 generates an image showing the change amount of the blood volume in the blood volume mode image 62, which is acquired by the subtraction processing by the calculation unit 59, with reference to the blood volume at a time point set by the user. The generation of the image is executed at a certain time interval set by the image acquisition unit 58. In addition, the time point that the user uses as a reference is based on a time at which the reference point image 67 is acquired.

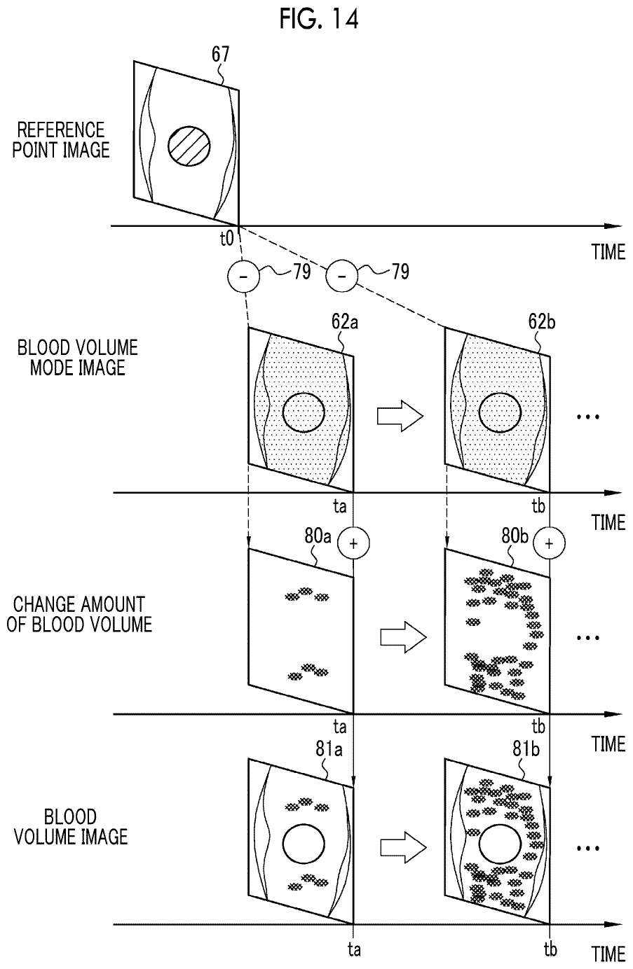

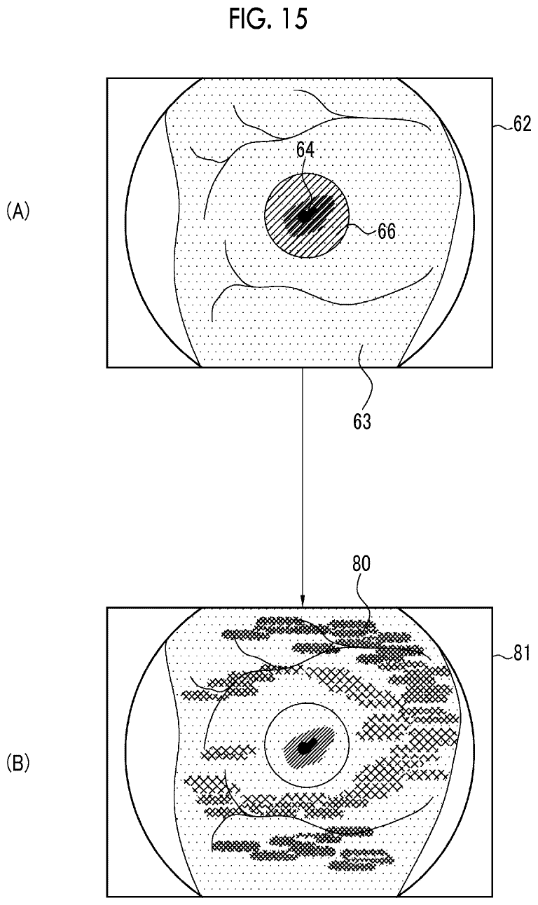

For the image showing the change amount of the blood volume, as shown in FIG. 14, in a case where the image acquisition unit 58 acquires the reference point image 67 and a blood volume mode image 62a, the calculation unit 59 executes subtraction processing 79, and a change amount 80a of the blood volume is calculated. Further, the time-series image generation unit 60 generates a blood volume image 81a by using the blood volume mode image 62a and the calculated change amount 80a of the blood volume. In addition, in a case where the user sets a certain time interval, the image acquisition unit 58 reacquires a blood volume mode image 62b, the calculation unit 59 executes the subtraction processing 79 by using the reference point image 67 to calculate a change amount 80b of the blood volume, and the time-series image generation unit 60 generates a blood volume image 81b by using the blood volume mode image 62b and the calculated change amount 80b of the blood volume. Thereafter, a series of flows is repeated.

It is preferable that the time-series image generation unit 60 performs processing of generating an image in which a color map with different shades is assigned according to the change amount with reference to the blood volume calculated in the subtraction processing and the color map is superimposed on the white light equivalent image NP2. For example, as shown in (A) of FIG. 15, in a case where the time-series image generation unit 60 sets the congestion site 64 as the region of interest 66 in the observation target 63 captured in the blood volume mode image 62 and executes the reference point imaging and the subtraction processing, as shown in (B) of FIG. 15, the time-series image generation unit 60 may generate a blood volume image 81 by assigning a color map 80 with different shades according to the change amount with reference to the blood volume after the subtraction processing and superimposing the color map 80 on the blood volume mode image 62.

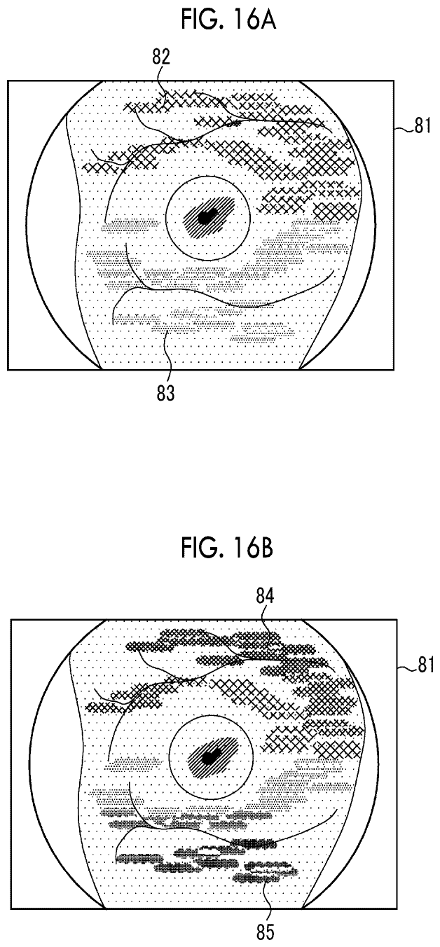

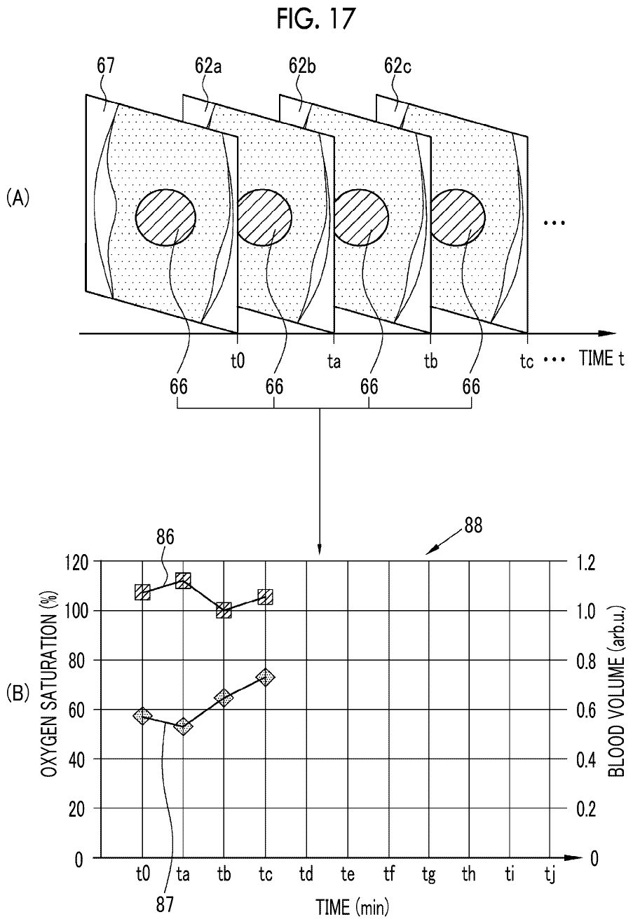

In addition, it is preferable that the time-series image generation unit 60 generates an image in which the change amount is superimposed on the white light equivalent image NP2 by representing a minus-side change and a plus-side change with different colors with reference to the blood volume calculated in the subtraction processing. For example, in the blood volume mode image 62 described above (see (A) of FIG. 15), as shown in FIG. 16A, a minus-side change 82 and a plus-side change 83 may be superimposed on the blood volume mode image 62 to generate the blood volume image 81 in which the blood volume is superimposed in different colors. The user may designate colors representing the minus-side change and the plus-side change with, for example, blue for the minus-side change, green for the plus-side change, or the like.

Further, the time-series image generation unit 60 may generate an image in which the minus-side change and the plus-side change are represented with different colors with respect to the above-described color map with different shades. For example, in the above-described color map 80 with different shades (see (B) of FIG. 15), as shown in FIG. 16B, a minus-side change 84 with different shades and a plus-side change 85 with different shades may be represented with different colors in the color map with different shades, and the blood volume image 81 in which the color map with different colors and different shades is superimposed may be generated.

The temporal change graph generation unit 61 generates a graph showing temporal changes of the blood volume and the oxygen saturation of the blood volume mode image 62 calculated by the calculation unit 59. The temporal change graph generation unit 61 performs control of executing the calculation processing based on the pixels in the same region as the observation target set as the region of interest for each image acquired by the image acquisition unit 58, and plotting the calculated blood volume and oxygen saturation on a graph to generate the graph, in displaying the graph. For example, as shown in (A) of FIG. 17, in the pixels included in the regions of interest 66 of the blood volume mode images 62a, 62b, and 62c acquired by the image acquisition unit 58 at certain time intervals ta, tb, and tc, the calculation unit 59 calculates an average value 86 of the blood volumes and an average value 87 of the oxygen saturations in the region of interest 66 of each blood volume image.

Regarding the average value 86 of the blood volumes and the average value 87 of the oxygen saturations of each blood volume image calculated as described above, for example, as shown in (B) of FIG. 17, in a case where the temporal change graph generation unit 61 generates a blood-volume-and-oxygen-saturation-at-each-time-point graph 88, the average values for the blood volume image obtained by the calculation are automatically plotted at each time point at which the image is acquired. Since the image is reacquired at a certain time interval set by the user, and the average value 86 of the blood volumes and the average value 87 of the oxygen saturations in the region of interest 66 of the image are plotted, the temporal changes of the blood volume and the oxygen saturation in the region of interest 66 of the observation target can be quantitatively ascertained.

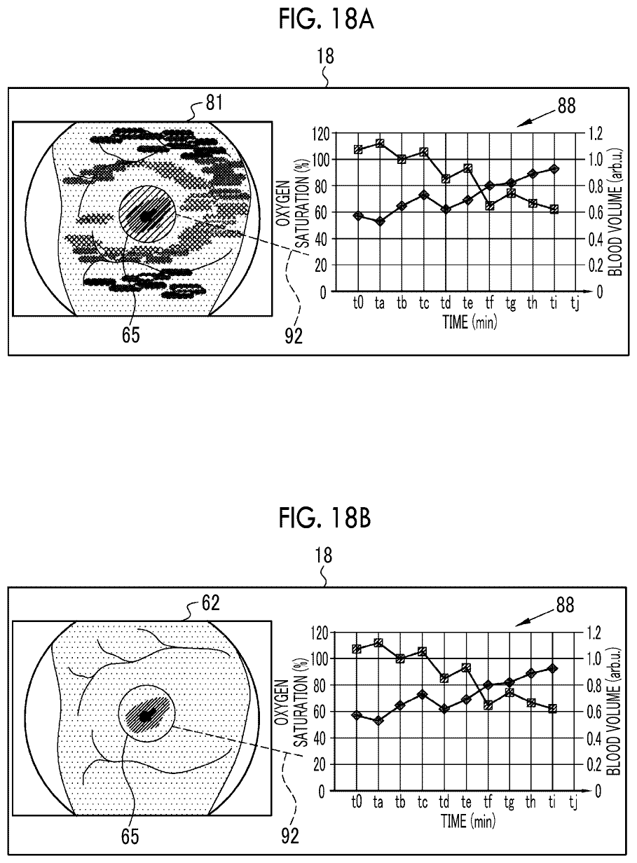

The blood volume image display unit 57 displays, on the display, at least one of an image indicating the change amount of the blood volume or the blood-volume-and-oxygen-saturation-at-each-time-point graph 88. For example, as shown in FIG. 18A, the blood volume image display unit 57 displays the region-of-interest target 65, the blood volume image 81, the blood-volume-and-oxygen-saturation-at-each-time-point graph 88, and a region-of-interest indication line 92 on the extended display 18. The region-of-interest target 65 and the region-of-interest indication line 92 indicate a region used for the calculation of the blood-volume-and-oxygen-saturation-at-each-time-point graph 88. In addition, in a case where only the graph is displayed, for example, as shown in FIG. 18B, the region-of-interest target 65, the blood volume mode image 62, the blood-volume-and-oxygen-saturation-at-each-time-point graph 88, and the region-of-interest indication line 92 are displayed on the extended display 18, and the blood volume image 81 on which the change amount of the blood volume is superimposed is not displayed.

In a case where the blood volume image 81 is not displayed, the blood volume image display unit 57 displays the blood volume mode image 62 instead of the blood volume image 81. Therefore, in a case where the graph is displayed, the region-of-interest target 65 and the region-of-interest indication line 92 of the observation target are always displayed, and it is possible to indicate to the user which region is used as the basis for the calculation of the blood-volume-and-oxygen-saturation-at-each-time-point graph 88.

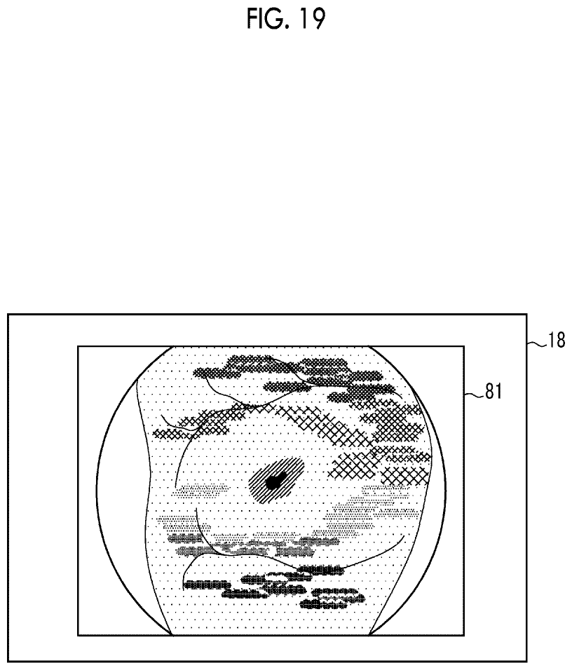

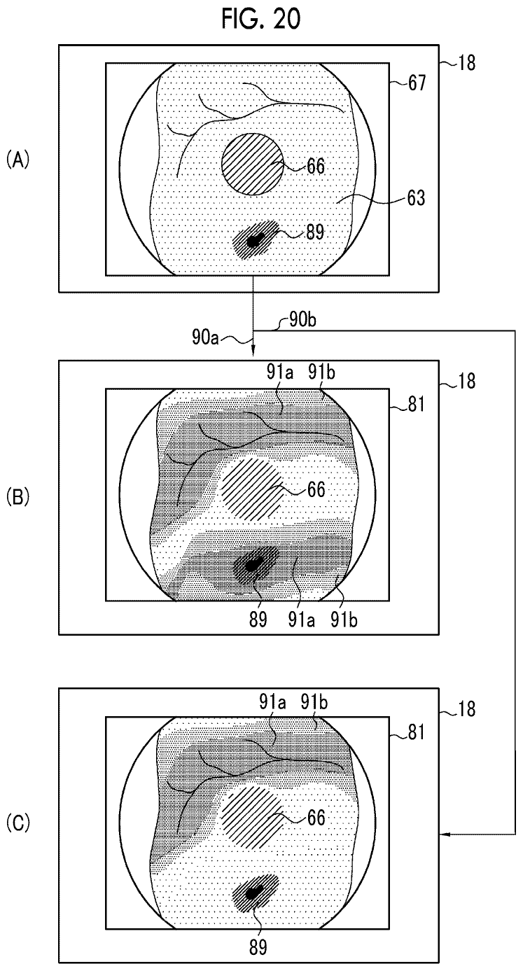

The blood volume image display unit 57 performs control of enabling the user to change display or non-display in the graph. For example, as shown in FIG. 19, the blood volume image display unit 57 may display only the blood volume image 81 on the extended display 18. In a case of non-display of the graph, the region-of-interest target 65 is not displayed, and visibility of the image (for example, the color map described above) indicating the change amount of the blood volume is further improved.

Hereinafter, examples using the above-described configuration will be described.

### Example 1

By performing the reference point imaging at a normal site, it is checked how much larger the blood volume of the observation target is than that of the normal site. For example, as shown in (A) of FIG. 20, in a case where the image acquisition unit 58 sets a normal site in the observation target 63 as the region of interest 66, executes the reference point imaging to acquire the reference point image 67, and further acquires the blood volume mode image 62, and the calculation unit 59 executes the subtraction processing on the normal site set as the region of interest 66, in a case where a doubtful site 89 shown in the generated blood volume image 81 is in a congestion state 90a, as shown in (B) of FIG. 20, regions 91a and 91b in which the change amount of the blood volume is larger than that in the region of interest 66 are represented around the doubtful site 89 shown in the blood volume image 81 and are superimposed on the white light equivalent image NP2 to be displayed on the extended display 18. In addition, in a case of a state 90b in which a difference in the blood volume between the doubtful site 89 and the region of interest 66 is small, as shown in (C) of FIG. 20, a region indicating the change amount of the blood volume between the doubtful site 89 shown in the blood volume image 81 and the region of interest 66 is not displayed on the extended display 18. In (B) and (C) of FIG. 20, the region of interest 66 is depicted as a region for the description of the drawings, but the region of interest 66 is not actually displayed on the extended display 18. In addition, the acquisition of the blood volume mode image can be executed by the user at any timing after the reference point imaging is executed.

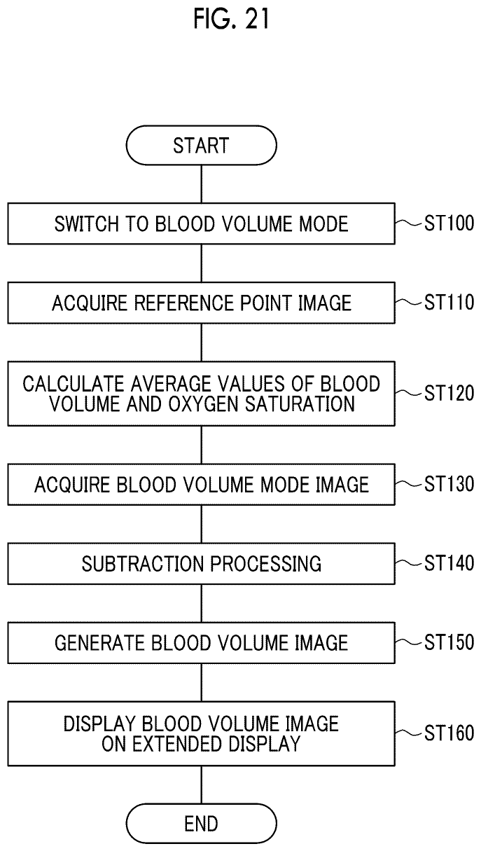

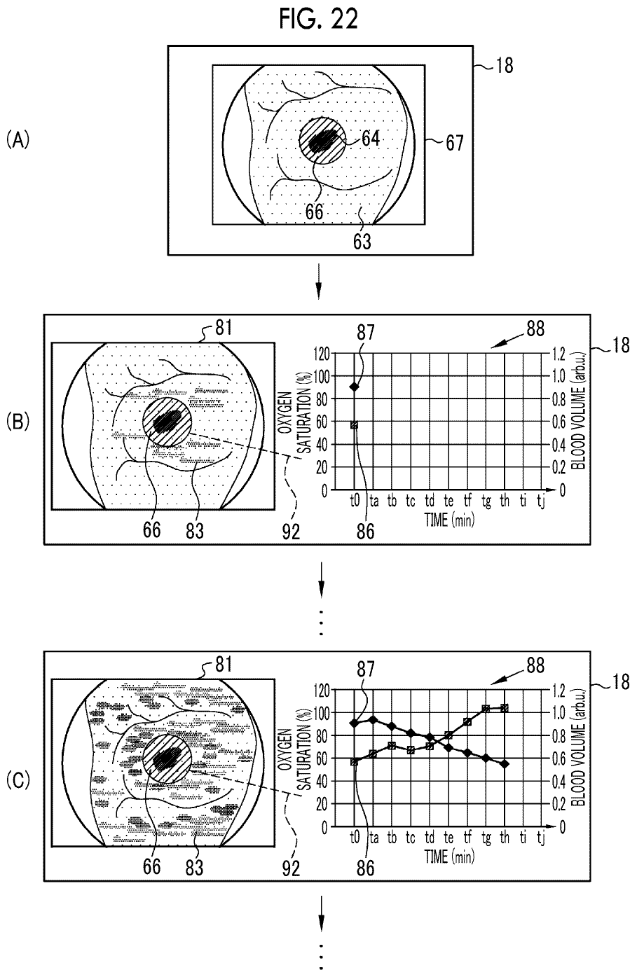

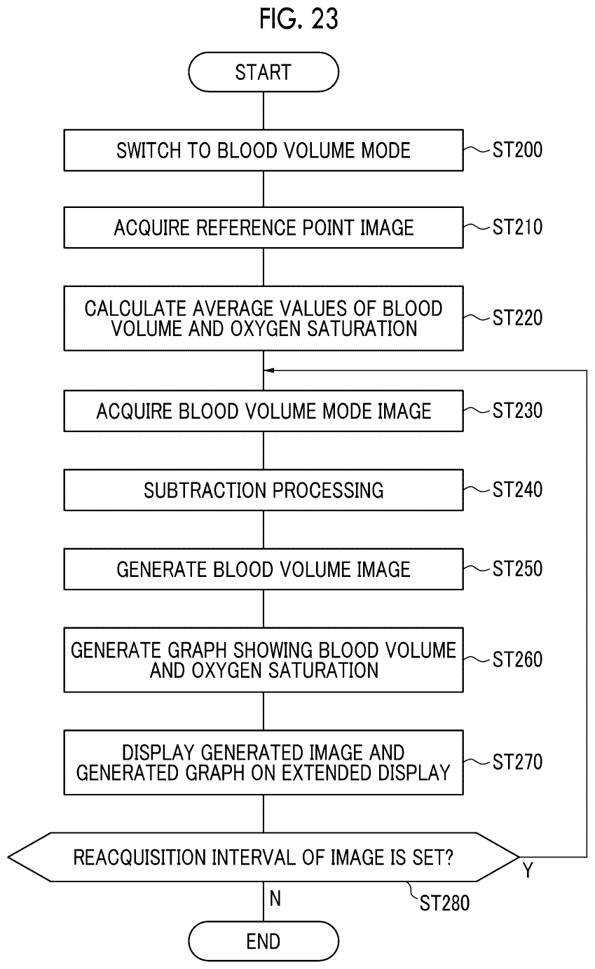

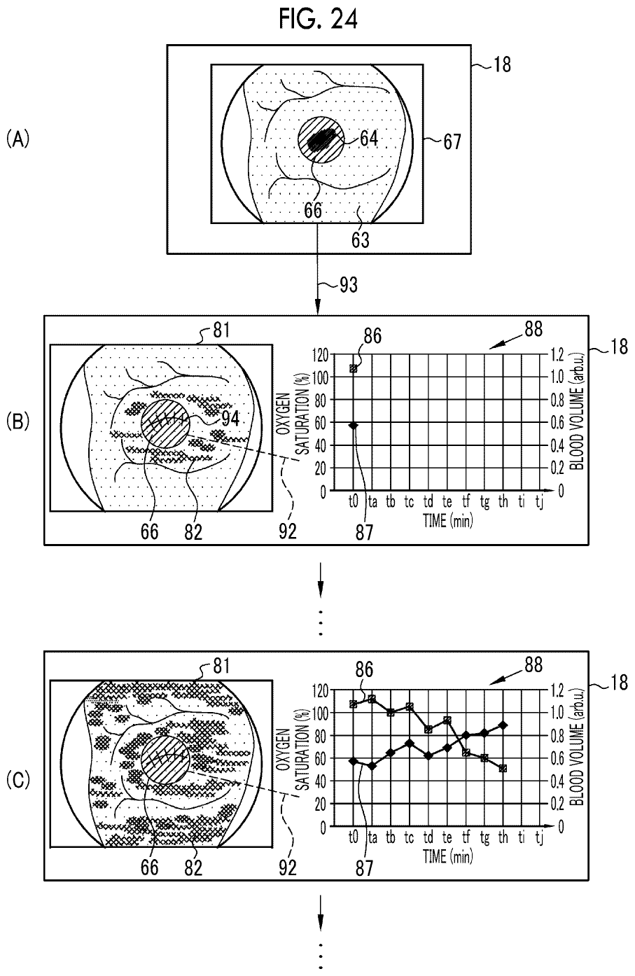

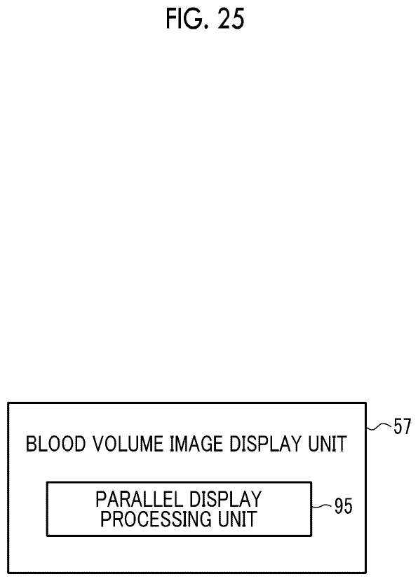

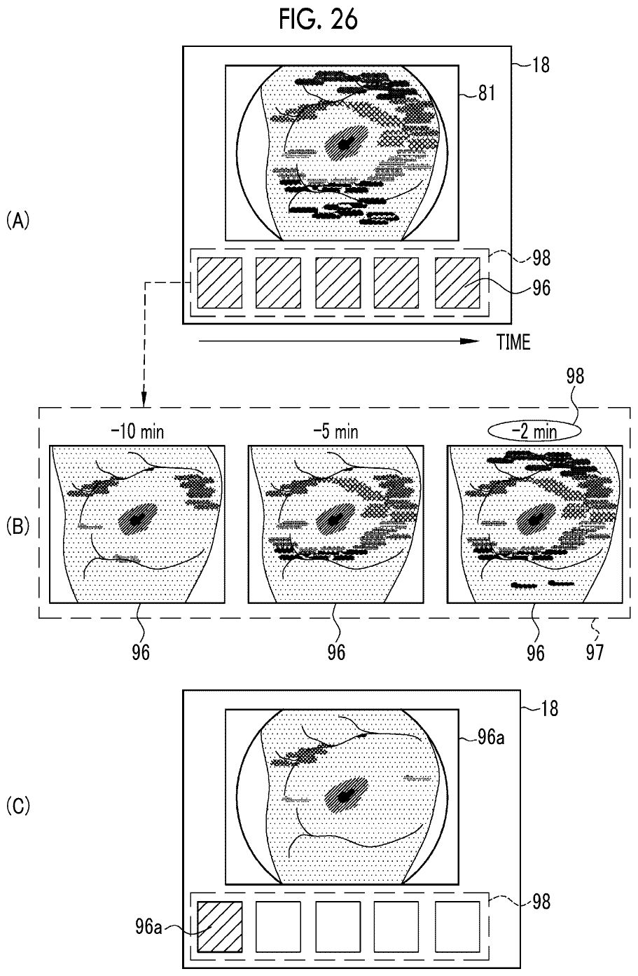

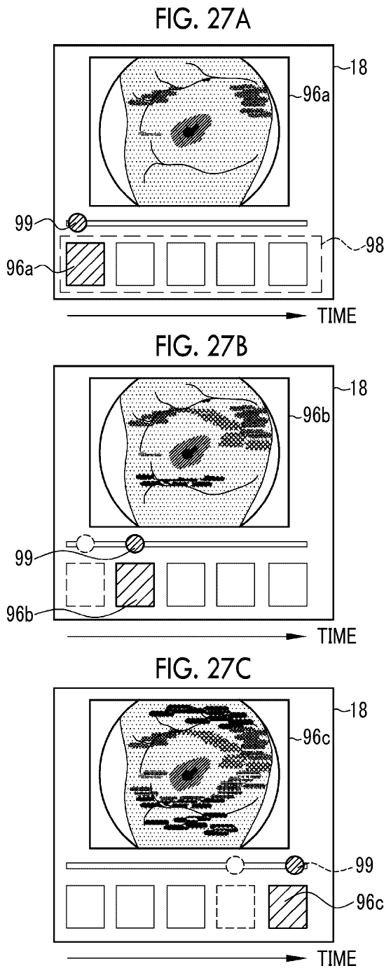

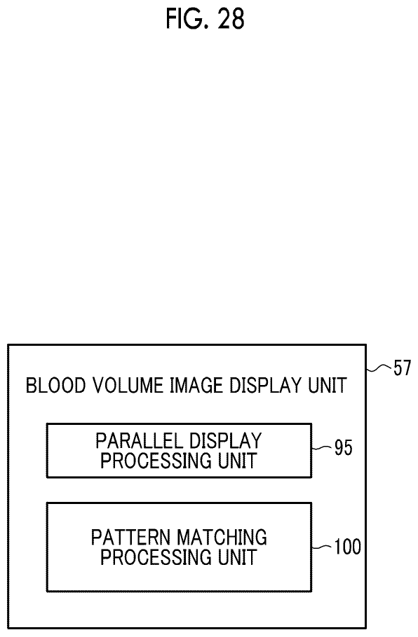

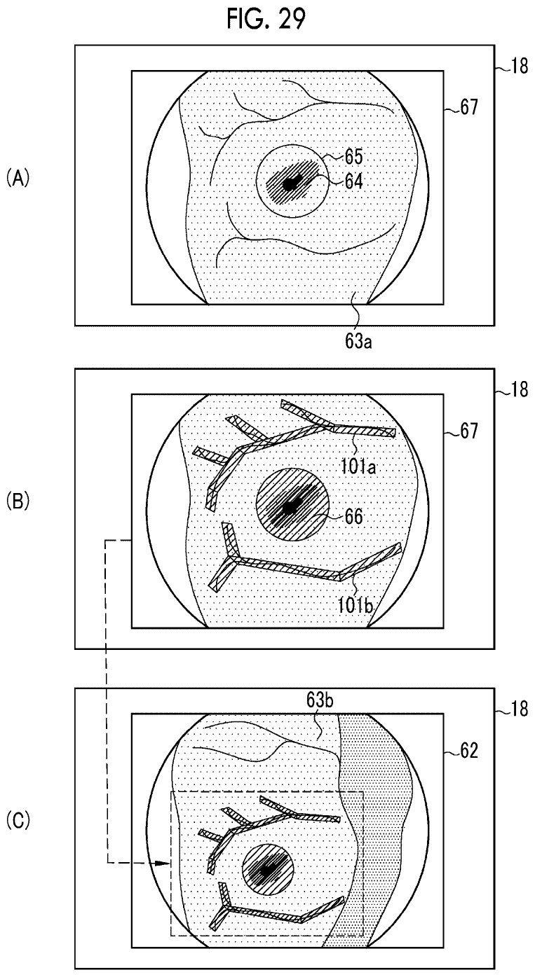

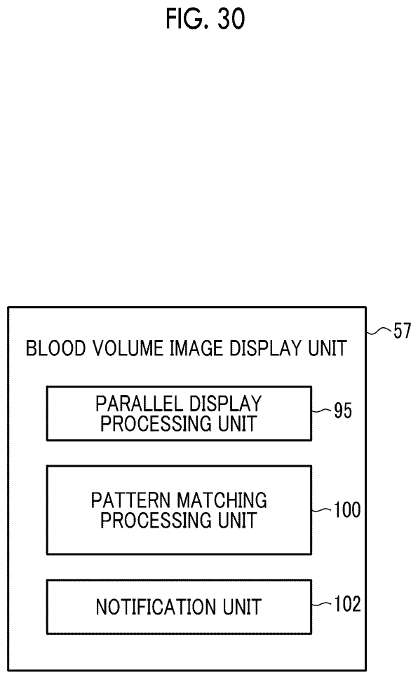

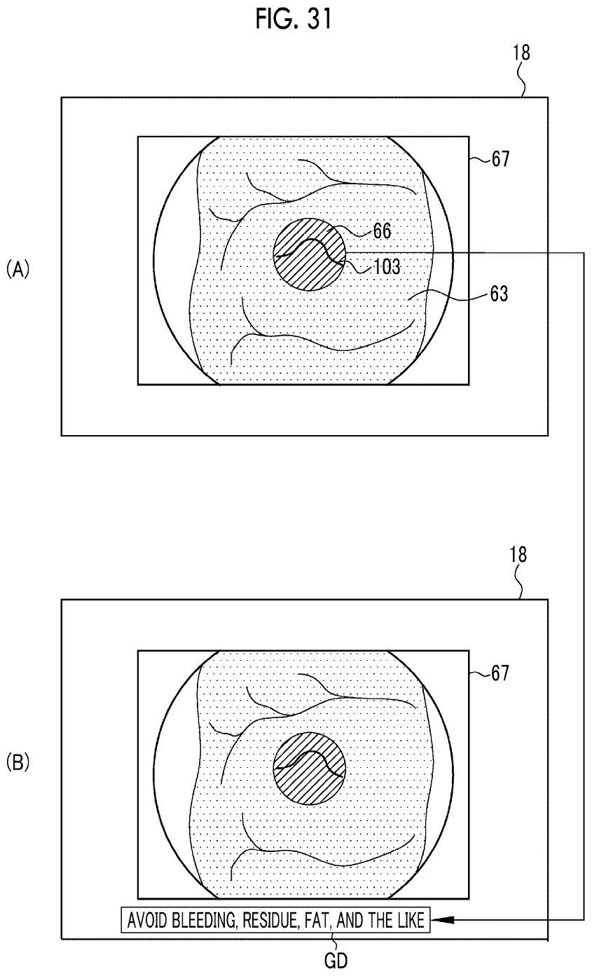

A flow of a series of processing in Example 1 by the endoscope system 10 will be described with reference to a flowchart of FIG. 21. Switching to the blood volume mode is made by automatic switching or the user operating the mode selector switch 12e (step ST100). The reference point image acquisition instruction switch 12f is pressed in a state in which the observation target used as a reference is included in the region-of-interest target 65, and the reference point imaging is executed to acquire the reference point image 67 using the image captured in the blood volume mode (step ST110). The calculation processing is executed to calculate the average values of the blood volume and the oxygen saturation as the reference values based on the pixels included in the region of interest 66 shown in the reference point image 67 (step ST120). The blood volume mode image is acquired at any timing after the reference point imaging (step ST130). The subtraction processing of subtracting the reference value of the blood volume obtained in the calculation processing from the blood volume calculated based on the pixels included in the entire blood volume mode image is executed (step ST140). The change amount of the blood volume due to the subtraction processing is superimposed on the white light equivalent image NP2 to generate the blood volume image (step ST150). The generated blood volume image is displayed on the extended display 18 (step ST160).

With the above-described configuration, in displaying the change amount of the blood volume, the endoscope system 10 can use the region of interest 66 as a reference, so that the user can designate the observation target and can visually recognize the change amount of the blood volume with reference to the observation target. In addition, it is possible to easily determine whether or not the observation target is in a congestion state.

### Example 2

The reference point imaging for setting a site where the congestion is checked as a region of interest is executed, an image is acquired at a time interval set by the user, and the blood volume and the oxygen saturation are calculated for each image. For example, as shown in (A) of FIG. 22, in a case where the image acquisition unit 58 sets the congestion site 64 in the observation target 63 as the region of interest 66, executes the reference point imaging to acquire the reference point image 67, and further acquires the blood volume mode image 62, and the calculation unit 59 executes the subtraction processing on the congestion site 64 set as the region of interest 66, as shown in (B) of FIG. 22, the time-series image generation unit 60 generates the blood volume image 81 by superimposing the plus-side change 83 of the blood volume on the blood volume mode image 62, and the temporal change graph generation unit 61 generates the blood-volume-and-oxygen-saturation-at-each-time-point graph 88 in which the average value 86 of the blood volume and the average value 87 of the oxygen saturation are plotted, so that the blood-volume-and-oxygen-saturation-at-each-time-point graph 88 is displayed on the extended display 18 by the blood volume image display unit 57. In a case where the user executes setting for acquiring the image at a certain time interval, as shown in (C) of FIG. 22, for each image reacquired at a certain time interval by the image acquisition unit 58, the time-series image generation unit 60 generates the blood volume image 81 by superimposing the plus-side change 83 of the blood volume on the blood volume mode image 62, and the temporal change graph generation unit 61 generates the blood-volume-and-oxygen-saturation-at-each-time-point graph 88 in which the average value 86 of the blood volume and the average value 87 of the oxygen saturation are plotted, so that the blood-volume-and-oxygen-saturation-at-each-time-point graph 88 is displayed on the extended display 18 by the blood volume image display unit 57. The display of the change amount of the blood volume is not limited to the plus-side change 83, and may be set by the user using the minus-side change 82, color maps with different shades, or the like to make it easier to visually recognize the change in the congestion state.

A flow of a series of processing in Example 2 by the endoscope system 10 will be described with reference to a flowchart of FIG. 23. Steps ST200 to ST250 are executed by the same processing as steps ST100 to ST150 in FIG. 21, and the description thereof will be omitted. In Example 2, further, a graph in which the average values of the blood volume and the oxygen saturation are plotted is generated (step ST260), and the generated blood volume image and graph are displayed on the extended display 18 (step ST270). In a case where the user executes setting for reacquisition of the image at a certain time interval (Y in step ST280), a procedure is performed again from the reacquisition of the blood volume mode image 62 at the set time interval (step ST230) and the subtraction processing of subtracting the reference value of the blood volume obtained in the calculation processing from the blood volume calculated based on the pixels included in the entire blood volume mode image (step ST240). In addition, in a case where the setting for reacquiring the image is not executed or is changed to setting for stopping the reacquisition of the image (N in step ST280), the blood volume mode image is not reacquired, and the blood volume image and the graph displayed on the extended display 18 are not updated.

With the above configuration, the endoscope system 10 can ascertain the passage of time with reference to the congestion site, so that it is possible to check whether or not the congestion state is progressing with the passage of time by limiting the site. Then, graphs showing the blood volume and the oxygen saturation at each time point are displayed in parallel, so that the oxygen saturation is ascertained at the same time as the blood volume, and it is possible to distinguishably determine whether a reference site is simply a site with a large blood volume or is in the congestion state in which the blood volume is large and oxygen is also consumed.

### Example 3

In a case where a surgical treatment is executed on a site where the congestion is checked such that the blood that is retained escapes to another path by executing a vascular treatment, it is checked that the blood that has been retained is reduced with the passage of time. Specifically, the reference point imaging is executed with reference to the congestion site before the vascular treatment, and after the vascular treatment of the congestion site, an operation of observing the same region over time is performed.

For example, as shown in (A) of FIG. 24, before the vascular treatment, the user performs processing of setting the congestion site 64 in the observation target 63 as the region of interest 66 and acquiring the reference point image 67 by executing the reference point imaging via the image acquisition unit 58. The user executes vascular treatment 93 on the congestion site 64 after the acquisition of the reference point image 67. After the vascular treatment 93 is executed, the user performs setting for acquiring the image at a certain time interval, and as shown in (B) of FIG. 24, in a case where the image acquisition unit 58 acquires the blood volume mode image 62, and the calculation unit 59 executes the subtraction processing on a vascular treatment site 94 set as the region of interest 66, the time-series image generation unit 60 superimposes the minus-side change 82 of the blood volume on the blood volume mode image 62 to generate the blood volume image 81, and the temporal change graph generation unit 61 generates the blood-volume-and-oxygen-saturation-at-each-time-point graph 88 in which the average value 86 of the blood volume and the average value 87 of the oxygen saturation are plotted, so that the blood volume and the oxygen saturation are displayed on the extended display 18 by the blood volume image display unit 57. Further, as shown in (C) of FIG. 24, the image acquisition unit 58 reacquires the blood volume mode image 62 with reference to the vascular treatment site 94 at a certain time interval set by the user. In addition, the time-series image generation unit 60 superimposes the minus-side change 82 of the blood volume on the blood volume mode image 62 with reference to the congestion site 64 on which the reference point imaging is executed to generate the blood volume image 81. Further, the temporal change graph generation unit 61 plots the average value 86 of the blood volume and the average value 87 of the oxygen saturation based on the reacquired blood volume mode image 62 to generate the blood-volume-and-oxygen-saturation-at-each-time-point graph 88. The blood volume image 81 and the blood-volume-and-oxygen-saturation-at-each-time-point graph 88 are displayed on the extended display 18 by the blood volume image display unit 57. The display of the change amount of the blood volume is not limited to the minus-side change 82, and may be set by the user using the plus-side change 83, color maps with different shades, or the like to make it easier to visually recognize the temporal change of the congestion site 64 after the vascular treatment.

A series of processing in Example 3 by the endoscope system 10 is the same as in Example 2 (see FIG. 23).

With the above configuration, the endoscope system 10 can ascertain the passage of time in the congestion state after the vascular treatment with reference to the congestion site, so that it is possible to check a decrease in the blood that has been retained in the congestion site with the passage of time. In addition, the graphs showing the blood volume and oxygen saturation at each time point are displayed in parallel, so that a rate of change can be quantitatively ascertained from a gradient of the graph. With the endoscope system 10, since the blood volume can be visualized by special light imaging, and a temporal change image from a certain reference point can be continuously recorded, a degree of progression of the congestion with reference to a designated site can be ascertained.

The blood volume image display unit 57 may comprise a parallel display processing unit 95 as shown in FIG. 25. The parallel display processing unit 95 performs control to convert images acquired by the image acquisition unit 58 into thumbnail images and to display the images in parallel in time series on the extended display.

For example, as shown in (A) of FIG. 26, the parallel display processing unit 95 may generate a thumbnail image 98 in which past images 96 acquired by the image acquisition unit 58 are arranged in time series at a lower part of a screen of the blood volume image 81. The thumbnail image 98 shows five past images 96 arranged side by side so as not to make the drawing complicated, but in practice, control of setting the number or form (for example, seven) that is easy for the user to see is performed by the parallel display processing unit 95. In addition, in the drawing, the reference numerals may be assigned to only some parts in order to prevent complications. In addition, the parallel display processing unit 95 may execute control of changing the number of the past images 96 arranged in time series and a position of the thumbnail image 98 according to setting of the user. For example, the user may execute setting for changing the position of the thumbnail image 98 to an upper part of the screen.

(B) of FIG. 26 is a diagram of the thumbnail image 98 in which some of the past images 96 are enlarged and displayed, and is an example of the past images 96 that are actually visually recognized by the user and that show the change amount of the blood volume, are converted into the thumbnail images, and are displayed in parallel in time series. The numerical value displayed at the upper part of the screen of the past image 96 indicates a past image acquisition time 97 in a case where a current time is used as a reference. The past image acquisition time 97 may be controlled to be displayed on some of the past images 96 (for example, displayed on a left end image and a center image), or may be changed by the user. In addition, in a case where the user changes the past image acquisition time 97, only a designated image may not be displayed, or the display of “−2 min” may be changed to a current time or the like (for example, 15:00).

In addition, as shown in (C) of FIG. 26, in a case where the user selects a past image 96a which is converted into a thumbnail image while being arranged in time series at the lower part of the screen of the blood volume image 81, the parallel display processing unit 95 may execute control of displaying the enlarged past image 96a on the extended display 18 via the blood volume image display unit 57. It is preferable that the parallel display processing unit 95 performs control of enabling the user to select all the acquired past images.

The blood volume images 81 are arranged in time series and converted into thumbnail images by the parallel display processing unit 95, so that the user can view a plurality of images arranged in time series at once, and can easily ascertain temporal progress of the change amount of the blood volume in the observation target.

In addition, the parallel display processing unit 95 may perform control of displaying a scroll bar for scrolling through the past images displayed in parallel as described above on the blood volume image. For example, as shown in FIG. 27A, in a case where the user displays, on the extended display 18, the past image 96a converted into a thumbnail image while being arranged in time series at the lower part of the screen of the blood volume image 81, and the past image 96a enlarged and displayed on the extended display 18, by means of the blood volume image display unit 57, the parallel display processing unit 95 may display a scroll bar 99 for transitioning (so-called scrolling) between the past images.

As shown in FIG. 27B, in a case where the scroll bar 99 transitions to the right, the parallel display processing unit 95 may perform control of switching from the past image 96a that is enlarged and displayed on the extended display 18 to a past image 96b arranged in parallel on the right side, and as shown in FIG. 27C, in a case where the scroll bar 99 transitions to a right end, the parallel display processing unit 95 may perform control of switching to a past image 96c arranged in parallel at the right end by enlarging and displaying the past image 96c on the extended display 18. The transition of the scroll bar 99 may be controlled to be automatically executed, or the user may transition the scroll bar 99 to switch the display of the past image. In addition, the past images which are switched by the transition of the scroll bar 99 are not limited to the five past images displayed on the extended display 18, and it is preferable to perform control of switching all the acquired past images by the transition of the scroll bar 99.

The parallel display processing unit 95 displays the scroll bar for scrolling through the past images displayed in parallel on the blood volume image, so that the user can view the past images by fast forwarding using the scroll bar, and can qualitatively ascertain the rate of the change in the blood volume.

In addition, the blood volume image display unit 57 may comprise a pattern matching processing unit 100 as in Modification Example 1 shown in FIG. 28. The pattern matching processing unit 100 performs pattern matching based on a blood vessel shape of the observation target in the reference point imaging, and performs control of executing registration in a case where the endoscope or the observation target moves.

For example, as shown in (A) of FIG. 29, in a case where the image acquisition unit 58 acquires the reference point image 67 by executing the reference point imaging on the congestion site 64 in an observation target 63a at a position within the region-of-interest target 65, the image acquisition unit 58 may recognize blood vessel shapes 101a and 101b in the observation target 63 and execute pattern matching for fixed-point observation of the region of interest 66, as shown in (B) of FIG. 29. It is preferable that the pattern matching is executed using illumination light B1, which is the first blue light, in order to make the blood vessel more clearly visible (see FIG. 7). Regarding the observation target 63a subjected to the pattern matching, as shown in (C) of FIG. 29, even in a case where, in the blood volume mode image 62, a position or a distance between the observation target 63a and the endoscope is changed, and the observation target 63b is imaged, the image acquisition unit 58 uses the blood vessel shapes 101a and 101b to ascertain a position of the region of interest 66, and acquires the blood volume mode image 62 such that the region of interest 66 is imaged.

The image acquisition unit 58 executes the registration based on the blood vessel shape in the observation target, so that the change amount of the blood volume and the oxygen saturation can be calculated for each pixel included in the region of interest at the same position even in a case where the endoscope or the subject moves. The pixel used for the calculation may be all the pixels included in the region of interest, or may be some of the pixels included in the region of interest, such as every one pixel or every two pixels.

In addition, the blood volume image display unit 57 may comprise a notification unit 102 as in Modification Example 2 shown in FIG. 30. The notification unit 102 performs control of notifying the user of operation guidance for resetting the region of interest in a case where the observation target that is affected by a disturbance is set as the region of interest in the reference point imaging.

For example, as shown in (A) of FIG. 31, in a case where the image acquisition unit 58 erroneously sets fat 103 in the observation target 63 as the region of interest 66 to acquire the reference point image 67 in the reference point imaging, operation guidance GD such as “avoid bleeding, residue, fat, and the like” may be displayed below the reference point image 67 as shown in (B) of FIG. 31. In addition, the user may be notified by a voice or the like.

The blood volume image display unit 57 can issue guidance even in a case where the user erroneously sets a disturbance such as fat as the region of interest, so that the user can be prevented from setting a site where the change amount of the blood volume or the oxygen saturation cannot be accurately calculated as the region of interest.

In the above-described embodiment, a hardware structure of a processing unit that executes various types of processing, such as the blood volume image processing unit 56, the blood volume image display unit 57, the image acquisition unit 58, the calculation unit 59, the time-series image generation unit 60, and the temporal change graph generation unit 61, is various processors as described below. The various processors include a central processing unit (CPU) that is a general-purpose processor that executes software (programs) to function as various processing units, a graphical processing unit (GPU), a programmable logic device (PLD) that is a processor capable of changing a circuit configuration after manufacture, such as a field-programmable gate array (FPGA), and an exclusive electric circuit that is a processor having a circuit configuration exclusively designed to execute various types of processing.

One processing unit may be composed of one of the various processors or may be composed of a combination of two or more processors of the same type or different types (for example, a combination of a plurality of FPGAs, a combination of a CPU and an FPGA, or a combination of a CPU and a GPU). In addition, a plurality of processing units may be configured by one processor. As an example in which the plurality of processing units are composed of one processor, first, there is a form in which one processor is composed of a combination of one or more CPUs and software, and this processor functions as the plurality of processing units, as represented by a computer, such as a client or a server. Second, there is a form in which a processor that implements functions of an entire system including a plurality of processing units with one integrated circuit (IC) chip is used, as represented by a system-on-chip (SoC) or the like. As described above, the various processing units are configured by using one or more of the various processors described above, as the hardware structure.

Further, the hardware structure of these various processors is, more specifically, an electric circuit (circuitry) having a form in which circuit elements, such as semiconductor elements, are combined. A hardware structure of a storage unit is a storage device such as a hard disc drive (HDD) or a solid-state drive (SSD).

### Additional Note 1

An endoscope system comprising a processor, in which the processor is configured to: execute image acquisition processing of acquiring an endoscopic image obtained by imaging an observation target at a time interval set in advance by a user; execute calculation processing of calculating a blood volume and an oxygen saturation for each image acquired in the image acquisition processing; generate an image showing a change amount of the blood volume for each image acquired in the calculation processing with reference to a blood volume at a time point set by the user; generate a graph showing temporal changes of the blood volume and the oxygen saturation for each image acquired in the calculation processing; and display at least one of the image showing the change amount of the blood volume or the graph showing the temporal changes of the blood volume and the oxygen saturation on a display.

### Additional Note 2

The endoscope system according to additional note 1, in which the processor is configured to perform reference point imaging of setting the observation target that is a reference as a region of interest in the image acquisition processing.

### Additional Note 3

The endoscope system according to additional note 1 or 2, in which the processor is configured to calculate average values of the blood volume and the oxygen saturation based on pixels included in the region of interest, and perform control of setting the calculated average values as reference values of the blood volume and the oxygen saturation.

### Additional Note 4

The endoscope system according to additional note 3, in which the processor is configured to perform subtraction processing of subtracting the reference value of the blood volume from the blood volume calculated in the calculation processing.

### Additional Note 5

The endoscope system according to additional note 4, in which the processor is configured to assign a color map with different shades according to the change amount with reference to the blood volume calculated in the subtraction processing, and generate an image in which the color map is superimposed on the image.

### Additional Note 6

The endoscope system according to additional note 4 or 5, in which the processor is configured to generate an image in which the change amount is superimposed on the image by representing a minus-side change and a plus-side change with different colors with reference to the blood volume calculated in the subtraction processing.

### Additional Note 7

The endoscope system according to additional note 3, in which the processor is configured to perform control of executing the calculation processing based on pixels in the same region as the observation target set as the region of interest for each image acquired in the image acquisition processing to generate the graph in which the calculated blood volume and oxygen saturation are plotted, in displaying the graph.

### Additional Note 8

The endoscope system according to additional note 1, in which the processor is configured to perform control of converting the image obtained by the image acquisition processing into a thumbnail image and displaying the image in parallel in time series on the display.

### Additional Note 9

The endoscope system according to additional note 8, in which the processor is configured to perform control of displaying a scroll bar for scrolling through the images displayed in parallel on the display.

### Additional Note 10

The endoscope system according to additional note 1, in which the processor is configured to perform control of enabling the user to change display or non-display in the graph.

### Additional Note 11

The endoscope system according to additional note 2, in which the processor is configured to perform pattern matching based on a blood vessel shape of the observation target in the reference point imaging, and perform control of executing registration in a case where an endoscope or the observation target moves.

### Additional Note 12

The endoscope system according to additional note 2, in which the processor is configured to perform control of notifying the user of operation guidance for resetting the region of interest in a case of setting the observation target, which is affected by a disturbance, as the region of interest in the reference point imaging.

### EXPLANATION OF REFERENCES

10: endoscope system
12: endoscope
12a: insertion part
12b: operating part
12c: bendable part
12d: tip part
12e: mode selector switch
12f: reference point image acquisition instruction switch
12g: zoom operation part
13: light source device
14: processor device
15: display
16: user interface
17: extended processor device
18: extended display
20: light source unit
20a: V-LED
20b: BS-LED
20c: BL-LED
20d: G-LED
20e: R-LED
21: light source processor
23: optical path combining unit
24: light guide
30: illumination optical system
31: imaging optical system
32: illumination lens
35: objective lens
36: imaging sensor
37: imaging processor
40: CDS/AGC circuit
41: A/D converter
45: DSP
50: image processing unit
51: image communication unit
52: display controller
53: central control unit
54: normal mode
55: blood volume mode
56: blood volume image processing unit
57: blood volume image display unit
58: image acquisition unit
59: calculation unit
60: time-series image generation unit
61: temporal change graph generation unit
62, 62a, 62b, 62c: blood volume mode image
63, 63a, 63b: observation target
64: congestion site
65: region-of-interest target
66: region of interest
67: reference point image
68: oxidized hemoglobin
69: reduced hemoglobin
70: reflected light in vicinity of 470 nm
71: reflected light in vicinity of 550 nm
72: reflected light in vicinity of 620 nm
73: signal ratio in short-wave region
74: signal ratio in long-wave region
75: LUT
76: change direction of oxygen saturation
77: change direction of blood volume
78: pixel
79: subtraction processing
80: color map with different shades
80a, 80b: change amount of blood volume
81, 81a, 81b: blood volume image
82: minus-side change
83: plus-side change
84: minus-side change with different shades
85: plus-side change with different shades
86: average value of blood volume
87: average value of oxygen saturation
88: blood-volume-and-oxygen-saturation-at-each-time-point graph
89: doubtful site
90a: congestion state
90b: state in which difference is small
91a, 91b: region in which change amount of blood volume is large
92: region-of-interest indication line
93: vascular treatment
94: vascular treatment site
95: parallel display processing unit
96, 96a, 96b, 96c: past image
97: past image acquisition time
98: thumbnail image
99: scroll bar
100: pattern matching processing unit
101a, 101b: blood vessel shape
102: notification unit
103: fat
NP1: white light image
NP2: white light equivalent image
BP: blood volume image
BP1: digestive tract internal blood volume image
BP2: serosal side blood volume image
GD: operation guidance
EL, ELL, ELH: contour line
ST100 to ST160, ST200 to ST280: step
t0, ta, tb, tc, td, te, tf, tg, th, ti, tj: time

What is claimed is:

1. An endoscope system comprising a processor,
wherein the processor is configured to:
execute image acquisition processing of acquiring an endoscopic image obtained by imaging an observation target at a time interval set in advance by a user;
execute calculation processing of calculating a blood volume and an oxygen saturation for each image acquired in the image acquisition processing;
generate an image showing a change amount of the blood volume for each image acquired in the calculation processing with reference to a blood volume at a time point set by the user;
generate a graph showing temporal changes in the blood volume and the oxygen saturation for each image acquired in the calculation processing; and
display, on a display, at least one of the image showing the change amount of the blood volume or the graph showing the temporal changes of the blood volume and the oxygen saturation.

2. The endoscope system according to claim 1,
wherein the processor is configured to perform reference point imaging by setting a reference observation target as a region of interest in the image acquisition processing.

3. The endoscope system according to claim 2,
wherein the processor is configured to calculate average values of the blood volume and the oxygen saturation based on pixels within the region of interest, and to set the calculated average values as reference values of the blood volume and the oxygen saturation.

4. The endoscope system according to claim 3,
wherein the processor is configured to perform subtraction processing by subtracting the reference value of the blood volume from the blood volume calculated in the calculation processing.

5. The endoscope system according to claim 4,
wherein the processor is configured to assign a color map with varying shades according to the change amount based on the blood volume calculated in the subtraction processing, and generate an image in which the color map is superimposed on the image.

6. The endoscope system according to claim 4,
wherein the processor is configured to generate an image in which the change amount is superimposed on the image by representing a minus-side change and a plus-side change with different colors, based on the blood volume calculated in the subtraction processing.

7. The endoscope system according to claim 3,
wherein the processor is configured to, for each image acquired in the image acquisition processing, perform the calculation processing based on pixels in the same region as the observation target set as the region of interest, and generate the graph in which the calculated blood volume and oxygen saturation are plotted, for displaying the graph.

8. The endoscope system according to claim 1,
wherein the processor is configured to perform converting the images obtained by the image acquisition processing into thumbnail images and displaying the thumbnail images in parallel in time series on the display.

9. The endoscope system according to claim 8,
wherein the processor is configured to perform displaying a scroll bar for scrolling through the images displayed in parallel on the display.

10. The endoscope system according to claim 1,
wherein the processor is configured to perform enabling the user to change between display or non-display of the graph.

11. The endoscope system according to claim 2,
wherein the processor is configured to perform pattern matching based on a blood vessel shape of the observation target in the reference point imaging, and perform registration in a case where an endoscope or the observation target moves.

12. The endoscope system according to claim 2,
wherein the processor is configured to perform notifying the user of operation guidance for resetting the region of interest in a case where the observation target affected by a disturbance is set as the region of interest in the reference point imaging.

13. An operation method for an endoscope system including a processor, the method comprising:
via the processor,
executing image acquisition processing of acquiring an endoscopic image obtained by imaging an observation target at a time interval set in advance by a user;
executing calculation processing of calculating a blood volume and an oxygen saturation for each image acquired in the image acquisition processing;
generating an image showing a change amount of the blood volume for each image acquired in the calculation processing with reference to a blood volume at a time point set by the user;
generating a graph showing temporal changes in the blood volume and the oxygen saturation for each image acquired in the calculation processing; and
displaying, on a display, at least one of the image showing the change amount of the blood volume or the graph showing the temporal changes of the blood volume and the oxygen saturation.

14. A non-transitory computer readable medium for storing a computer-executable program for causing a computer to function as an endoscope system, the computer-executable program causing a computer to implement:
a function of executing image acquisition processing of acquiring an endoscopic image obtained by imaging an observation target at a time interval set in advance by a user;
a function of executing calculation processing of calculating a blood volume and an oxygen saturation for each image acquired in the image acquisition processing;
a function of generating an image showing a change amount of the blood volume for each image acquired in the calculation processing with reference to a blood volume at a time point set by the user;
a function of generating a graph showing temporal changes in the blood volume and the oxygen saturation for each image acquired in the calculation processing; and
a function of displaying, on a display, at least one of the image showing the change amount of the blood volume or the graph showing the temporal changes of the blood volume and the oxygen saturation.
